(19) United States
(12) Patent Application Publication
(10) Pub. No.: US 2023/0218684 A1
(43) Pub. Date: Jul. 13, 2023

(54) ENGINEERED BACTERIA FOR USE IN VACCINE COMPOSITIONS

(71) Applicants: NORTH CAROLINA STATE UNIVERSITY, Raleigh, NC (US); COLORADO STATE UNIVERSITY RESEARCH FOUNDATION, Fort Collins, CO (US)

(72) Inventors: Rodolphe Barrangou, Raleigh, NC (US); Gregg A. Dean, Fort Collins, CO (US)

(21) Appl. No.: 17/995,069

(22) PCT Filed: Mar. 29, 2021
(86) PCT No.: PCT/US2021/024629

§ 371 Date: Sep. 29, 2022

Related U.S. Application Data
(60) Provisional application No. 63/002,260, filed on Mar. 30, 2020.

(51) Int. Cl. A61K 35/744; C12N 1/20; C12N 15/74; C07K 14/165; A61P 31/14; C07K 14/14

(52) U.S. Cl. CPC A61K 35/744; C12N 1/20; C12N 15/746; C07K 14/165; A61P 31/14; C07K 14/14; A61K 2039/523

(57) ABSTRACT

The present disclosure provides materials and methods related to engineered bacteria for use in vaccines. In particular, the present disclosure provides novel compositions and methods for generating vaccine compositions comprising bacteria (e.g., Lactobacillus) engineered to express immunogenic polypeptides and immunogenicity-enhancing adjuvant polypeptides to treat and/or prevent infection from a pathogenic organism (e.g., coronavirus).

### CROSS REFERENCE TO RELATED APPLICATIONS

This application claims priority to and the benefit of U.S. Provisional Patent Application No. 63/002,260 filed Mar. 30, 2020, which is incorporated herein by reference in its entirety for all purposes.

### GOVERNMENT SUPPORT

This invention was made with government support under R01 AI141603 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 28,380 Byte ASCII (Text) file named “2021-03-29_39278-601_ SQL_ST25” created on Mar. 29, 2021.

### FIELD

The present disclosure provides materials and methods related to engineered bacteria for use in vaccines. In particular, the present disclosure provides novel compositions and methods for generating vaccine compositions comprising bacteria (e.g., Lactobacillus) engineered to express immunogenic polypeptides and immunogenicity-enhancing adjuvant polypeptides to treat and/or prevent infection from a pathogenic organism (e.g., coronavirus).

### BACKGROUND

The relationship between the microbiome and its host has been rigorously studied during the last decade resulting in evidence supporting its role in health and disease. Research has shown that the microbiome is dependent on diet and on its environment and can be modulated by use of antibiotics, probiotics and/or prebiotics. Additionally, it is clear that the microbiome greatly influences mucosal health, but how vaccines, and any subsequent mucosal immune response, influence the microbiome is poorly understood. There is increasing evidence that the immunogenicity and efficacy of current vaccines are related to the intestinal microbiome. Multiple studies have also shown that probiotic administration prior to or concurrent with vaccination enhances B cell and antibody responses and provides the mucosa with direct protection from infection through interactions with the innate immune system. Trials of both parenteral and nonparenteral vaccines, in conjunction with probiotic administration, also point to probiotic bacteria as adjuvants. The mechanisms behind this phenomenon are incompletely understood but are likely due to probiotic surface structures. This inherent adjuvanticity has led to the use of probiotics, typically lactic acid bacteria, as nonparenteral live mucosal vaccine vectors. For example, probiotic strains of Lactobacillus are promising as oral vaccine vectors because of their ability for immune stimulation (e.g., binding to dendritic cells), their acid and bile tolerance, and their ability to express mucus-binding proteins and association with the mucosal epithelium.

### SUMMARY

Embodiments of the present disclosure include engineered bacterial cells comprising an exogenous polypeptide(s), and at least one adjuvant polypeptide, which are expressed by the bacterial cell. In accordance with these embodiments, the expression of the exogenous polypeptide(s) and the at least one adjuvant polypeptide can induce an immune response in a host to treat and/or prevent infection from a pathogenic organism.

In some embodiments, the engineered bacterial cell includes at least one of the following genera: Bacillus, Bacteroides, Bifidobacterium, Brevibacteria, Clostridium, Enterococcus, Escherichia coli, Lactobacillus, Lactococcus, Staphylococcus and Streptococcus. In some embodiments, the engineered bacterial cell includes at least one of the following species: Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus crispatus, Lactobacillus gasseri, Lactobacillus johnsonii, Lactobacillus paracasei, Lactobacillus plantarum, Lactobacillus reuteri, Lactobacillus rhamnosus, Lactobacillus salivarius Lactobacillus fermentum, Lactobacillus delbrueckii, and Lactococcus lactis. In some embodiments, the engineered bacterial cell includes at least one of the following strains: L. acidophilus NCFM, L. acidophilus La-14, L. casei Lc11, L. crispatus NCK 1350, L. crispatus NCK 1351, L. crispatus DNH-429, L. gasseri ATCC 33323, L. gasseri NCK 1338, L. gasseri NCK 1340, L. gasseri NCK 1341, L. gasseri NCK 1342, L. gasseri NCK 1343, L. gasseri Lg-36, L. gasseri NCK2140, L. gasseri NCK2141, L.gasseri JV V03, L. plantarum Lp-115, L. johnsonii NCK948, L. johnsonii NCK957, L. johnsonii NCK964, L. johnsonii NCK979, L. johnsonii NCK1370, L. johnsonii NCK2677, L. johnsonii NCC 533 L. plantarum Lpc-37, L. plantarum Lp115, L. rhamnosus HN001, L. rhamnosus GG, L. rhamnosus Lr-32, L. reuteri 1E1, L. salivarius Ls-33, L. salivarius NCK1352, L. salivarius NCK1355, B. lactis BL-04, B. lactis Bb-02, B. lactis B1-04, B. lactis Bi-07, B. breve Bb-03, B. bifidum Bb-06, B. longum B1-05, B. longum sp infantis Bi-26.

In some embodiments, the exogenous polypeptide is immunogenic. In some embodiments, the exogenous polypeptide is expressed on the surface of the bacterial cell, expressed in the cytosol of the bacterial cell, and/or secreted by the bacterial cell.

In some embodiments, the exogenous polypeptide is an antigen from a pathogenic organism. In some embodiments, the pathogenic organism is a bacteria or a virus. In some embodiments, the exogenous polypeptide is a rotavirus antigen. In some embodiments, the rotavirus antigen is selected from the group consisting of VP3, VP4, VP5, VP6, VP7, VP8, and any derivatives or fragments thereof.

In some embodiments, the exogenous polypeptide is a coronavirus antigen. In some embodiments, the exogenous polypeptide is a SARS-CoV-2 antigen. In some embodiments, the exogenous polypeptide is a feline enteric coronavirus antigen. In some embodiments, the coronavirus antigen is derived from a nucleocapsid protein (N), a spike protein (S), an envelope protein (E), or a membrane protein (M).

In some embodiments, the at least one adjuvant polypeptide enhances immunogenicity of the exogenous polypeptide. In some embodiments, the at least one adjuvant polypeptide targets the bacterial cell to a host cell, activates a cellular pathway in a host cell, and/or mimics a host cell immune factor. In some embodiments, the at least one adjuvant polypeptide is exogenous to the bacterial cell. In some embodiments, the at least one adjuvant polypeptide is endogenous to the bacterial cell. In some embodiments, the at least one adjuvant polypeptide is expressed on the surface of the bacterial cell, expressed in the cytosol of the bacterial cell, and/or secreted by the bacterial cell. In some embodiments, the at least one adjuvant polypeptide is flagellin (FliC) and/or type 1 fimbrin D-mannose specific adhesin protein (FimH), and any derivatives or fragments thereof.

In some embodiments, the at least one adjuvant polypeptide is a pro-inflammatory cytokine. In some embodiments, the at least one adjuvant polypeptide targets a dendritic cell (DC).

In some embodiments, the exogenous polypeptide and the at least one adjuvant polypeptide are co-expressed. In some embodiments, the exogenous polypeptide and/or the at least one adjuvant polypeptide are expressed as fusion polypeptides. In some embodiments, the exogenous polypeptide and/or the at least one adjuvant polypeptide are integrated into an S-layer protein (SLP) or an S-layer associated protein (SLAP).

In some embodiments, the cell further comprises at least a second exogenous polypeptide. In some embodiments, the exogenous polypeptide and the at least second exogenous polypeptide are from the same organism. In some embodiments, the exogenous polypeptide and the at least second exogenous polypeptide are from different organisms.

Embodiments of the present disclosure also include a vaccine composition comprising any of the engineered bacterial cells described herein.

Embodiments of the present disclosure also include a food product comprising any of the engineered bacterial cells described herein.

Embodiments of the present disclosure also include methods of inducing an immune response in a subject by administering a composition comprising any of the engineered bacterial cells described herein.

In some embodiments, the composition induces a mucosal and systemic immune response against the exogenous polypeptide. In some embodiments, the composition increases immunoglobulin A (IgA) antibodies specific for the exogenous polypeptide. In some embodiments, the composition increases immunoglobulin G (IgG) antibodies specific for the exogenous polypeptide.

### BRIEF DESCRIPTION OF THE DRAWINGS

FIG. 1: Diagram of peptide insertion into SlpA via homologous recombination. The SlpA gene was cloned and the virus peptide was inserted into a region of the sequence known to preserve SlpA structure and provide surface exposure. The plasmid was transfected into L. acidophilus and selected for homologous recombination using nutrient selective pressure.

FIGS. 2A-2B: Expression and validation of FliC. Flow cytometric analysis shows expression of FliC on the surface of L. acidophilus (A). HEK293T cell lines expressing a single receptor (TLR2, TLR5 or NOD2) were used to show that wild-type L. acidophilus (violet) activates TLR2 and NOD2, while the FliC-expressing bacteria (orange) were shown to activate TLR2, NOD2 and TLR5 (B). TLR5 specifically recognizes FliC thus adding functionality to the recombinant L. acidophilus. TLR, toll-like receptor; NOD2, Nucleotide-binding oligomerization domain-containing protein 2.

FIGS. 3A-3C: rLA PP uptake and MLN trafficking. ELISA plates were coated with murine GP2 and binding of GAD31 (LA.MPER) and GAD40 (LA.FimH.MPER) was determined. A significantly higher number of GAD40 bound to GP2 (A). GAD31 or GAD40 were directly inoculated into an isolated section of small intestine in anesthetized Balb/cJ mice. One hour later PP and MLN were collected and processed into single cell suspensions and plated on erythromycin resistance plates. CFU per 10⁶ live cells were calculated. There was a significant increase in GAD40 MLN trafficking compared to GAD31 (B). A similar increase in MLN trafficking was observed following oral delivery of cell trace violet labeled LA with GAD40 compared to GAD31 (not shown). Analysis of antigen presenting cells (APC) associated with cell trace violet labeled LA showed that GAD40 was more likely to be associated with MHCII⁺ cells than GAD 31 especially CD103⁺ dendritic cells (D). **p<0.05; *p<0.1 based on a Kruskal-Wallis one-way ANOVA. Macrophages: MHCII⁺CD64⁺; dendritic cells (DC): MHCII⁺CD64⁻CD11c⁺CD103^{+/-} CFU: colony forming units; PP: Peyer's patch; MLN: mesenteric lymph node; LA: Lactobacillus acidophilus.

FIGS. 4A-4B: Adjuvant effect of FliC mid FimH. FliC and FimH result in increased antigen-specific IgA producing cells in the mucosal lamina propria as measured by ELISPOT (A) and serum IgG as measured by ELISA (B).

FIGS. 5A-5C: Validation of rotavirus constructs. LA surface expression of VP8 10 amino acid peptide (VP8pep) (A), Salmonella spp. FliC (B), and E. coli FimH (C). LA surface expression was assessed by flow cytometry using anti-VP8pep, anti-FliC, and anti-FimH antibodies. All strains showed >95% expression.

FIG. 6: VP8-1 expression in cytosol of L. acidophilus. Bacteria were grown to exponential phase (exp) or overnight (ON, plateau phase), then disrupted with either lysozyme or with bead beating. Recombinant VP8 was used as a positive control (Lane 2) and wild-type LA (LA WT) served as negative controls (Lanes 3, 5, 7, 9). Arrows indicate VP8 expression in cultures disrupted by the bead beating method (Lanes 8 and 10). Molecular weight markers are in Lanes 1 and 11.

FIG. 7: Epitope specific B cells from the spleen of orally dosed Balb/c mice. Eight Balb/c mice (4 male and 4 female) were orally gavaged with buffer (soy trypsin inhibitor in bicarbonate buffer), NCK56 (Lactobacillus acidophilus without adjuvants and epitopes) or 3 or 5 times with GAD85 (Lactobacillus acidophilus expressing VP8_10AA in the surface layer protein slpA, VP8-1 in the cytoplasm, and FimH and FliC on the surface). Mice were euthanized by CO₂ asphyxiation and spleens collected and processed into single cell suspensions by physical dissociation. Approximately 500,000 cells were incubated for 18 hours on MultiScreen HTS IP Filter Plates (MiliporeSigma) coated overnight with VP8_10 AA peptide (left) or VP8-1 protein (right). Plates were developed using either IgG/IgA colorimetric or double-color FluoroSpot assay (ImmunoSpot). Spots were read by ImmunoSpot S6 Universal Analyzer (ImmunoSpot). Groups with a p-value less than 0.05 are shown (one-way non-parametric ANOVA (GraphPad Prism 8.1.0).

FIG. 8: Surface expression of adjuvants and SARS-CoV-2 peptides. The S1/S2 and S2′ peptides and FimH were inserted into the chromosome using a double-crossover homologous recombination-based gene replacement system. FliC was expressed from a plasmid. Surface expression of each recombinant protein is demonstrated by flow cytometry.

FIGS. 9A-9D: CRISPR-Cas9 gene editing of L. acidophilus. A pJG-based CRISPR-SpyCas9n plasmid encompassing the SpyCas9^{D10A} nickase mutant, the necessary L. acidophilus-compatible pORI-based replication machinery with a chloramphenicol-resistance marker and a guide RNA (green) targeting the gene of interest (LBA0447, encoding the lipoteichoic acid synthase), as well as a recombination template designed to generate a deletion (A). Screening of surviving colonies using PCR primers targeting the LBA0447 gene, showing the loss of the large WT band, and the presence of a shorter amplicon reflecting the deletion (B). Sequencing-based confirmation of the junction fragments generated by the CRISPR-based engineering (C). RNA-seq analysis of transcripts in the screened mutant showing the loss of LBA0447 transcription (D).

FIGS. 10A-10C: Cell surface composition of Lactobacillus acidophilus NCFM. A representative diagram presenting the various layers of the L. acidophilus surface, including the S-layer associated proteins (SLAPs) which are fused to FliC and FimH adjuvants (A). Diversity and relative amounts of various S-layer associated proteins in both log and stationary phases of growth (B). Quantitative proteomic analysis of SLAPs showing select proteins highly detected regardless of the growth phase. The green box outlines SLAPs of primary interest (C).

FIG. 11: Impact of different rLA vaccine constructs on fecal microbiome as measured by Shannon diversity (abundance and evenness).

FIG. 12: Nonmetric Multidimentional Scaling plots showing putative shift in the microbial community structure from time 0 to 10 (numbers within the ovals) under the wild type, MPER, and IL1b treatments; no particular shift pattern was observed under the FliC and negative control treatments.

FIG. 13: FACS histogram of IgA coated bacteria (lower panel) compared to a negative control run (upper panel) utilizing a sorting protocol.

FIG. 14: Representative schematic diagram of the constructs used in the various embodiments of the present disclosure (see also Table 1).

### DETAILED DESCRIPTION

Embodiments of the present disclosure provide materials and methods related to engineered bacteria for use in vaccines. In particular, the present disclosure provides novel compositions and methods for generating vaccine compositions comprising bacteria (e.g., Lactobacillus) engineered to express immunogenic polypeptides and immunogenicity-enhancing adjuvant polypeptides to treat and/or prevent infection from a pathogenic organism (e.g., coronavirus). In accordance with these embodiments, the present disclosure provides a vaccine platform comprising genetically engineered microorganisms for use in the production of pharmaceutical vaccine compositions, as well as methods of treating and/or preventing diseases and disorders associated with infections from pathogenic organisms. In some embodiments, the recombinant bacteria disclosed herein can be engineered to induce an immunogenic response in a host against any pathogenic organism using any immunogenic polypeptide. The recombinant bacteria generated using the vaccine platform technology of the present disclosure are safe, well tolerated, and augment the innate activities of a subject's immune response to achieve a therapeutic effect.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “animal” refers to any animal (e.g., a mammal), including, but not limited to, humans, non-human primates, pigs, rodents (e.g., mice, rats, etc.), flies, and the like.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “microorganism” refers to an organism or microbe of microscopic, submicroscopic, or ultramicroscopic size that typically consists of a single cell. Examples of microorganisms include bacteria, yeast, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus.

As used herein, “non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria, which are present in the indigenous microbiota of the gut. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Streptococcus and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus crispatus, Lactobacillus gasseri, Lactobacillus johnsonii, Lactobacillus paracasei, Lactobacillus plantarum, Lactobacillus reuteri, Lactobacillus rhamnosus, Lactobacillus salivarius Lactobacillus fermentum, Lactobacillus delbrueckii, and Lactococcus lactis. Naturally pathogenic bacteria may be genetically engineered to provide reduce or eliminate pathogenicity.

As used herein, the term “probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram positive bacteria. Some species, strains, and/or subtypes of non-pathogenic bacteria are currently recognized as probiotic bacteria. Examples of probiotic bacteria include, but are not limited to certain strains belonging to the genus Bifidobacteria, Escherichia, Lactobacillus, and Streptococcus, e.g., Bifidobacterium bifidum, Enterococcus faecium, Escherichia coli, Escherichia coli strain Nissle, Lactobacillus acidophilus, Lactobacillus crispatus, Lactobacillus gasseri, Lactobacillus bulgaricus, Lactobacillus paracasei, Lactobacillus plantarum, Lactobacillus reuteri, Lactobacillus rhamnosus, Lactobacillus salivarius, Lactobacillus fermentum, and Lactobacillus delbrueckii. In some embodiments, examples of strains include, but are not limited to, L. acidophilus NCFM, L. acidophilus La-14, L. casei Lc11, L. crispatus NCK 1350, L. crispatus NCK 1351, L. crispatus DNH-429, L. gasseri ATCC 33323, L. gasseri NCK 1338, L. gasseri NCK 1340, L. gasseri NCK 1341, L. gasseri NCK 1342, L. gasseri NCK 1343, L. gasseri Lg-36, L. gasseri NCK2140, L. gasseri NCK2141, L.gasseri JV V03, L. plantarum Lp-115, L. johnsonii NCK948, L. johnsonii NCK957, L. johnsonii NCK964, L. johnsonii NCK979, L. johnsonii NCK1370, L. johnsonii NCK2677, L. johnsonii NCC 533 L. plantarum Lpc-37, L. plantarum Lp115, L. rhamnosus HN001, L. rhamnosus GG, L. rhamnosus Lr-32, L. reuteri 1E1, L. salivarius Ls-33, L. salivarius NCK1352, L. salivarius NCK1355, B. lactis BL-04, B. lactis Bb-02, B. lactis B1-04, B. lactis Bi-07, B. breve Bb-03, B. bifidum Bb-06, B. longum B1-05, B. longum sp infantis Bi-26, or any combination thereof.

The probiotic may be a variant or a mutant strain of bacterium. Nonpathogenic bacteria may be genetically engineered to enhance or improve desired biological properties, e.g., survivability. Non-pathogenic bacteria may be genetically engineered to provide probiotic properties. Probiotic bacteria may be genetically engineered or programmed to enhance or improve probiotic properties.

As used herein, the term “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, a “non-native” nucleic acid sequence refers to a nucleic acid sequence not normally present in a bacterium, e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence. The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions. In some embodiments, the genetically engineered bacteria of the invention comprise a gene encoding an immunogenic polypeptide or antigenic polypeptide that is operably linked to a directly or indirectly inducible promoter that is not associated with said gene in nature.

As used herein, “operably linked” refers a nucleic acid sequence encoding an immunogenic polypeptide or antigenic polypeptide that is joined to a regulatory region sequence in a manner which allows expression of the nucleic acid sequence. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

As used herein, “promoter” refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. COMPOSITIONS AND METHODS

Emergence of novel human coronaviruses from animal reservoirs has likely been ongoing throughout the history of humanity. Only recently has this garnered more attention because of technological advances to detect new viruses and the devastating potential of rapid spread achieved by global movement of people. Despite persistent efforts, there is no vaccine platform available that can be quickly and efficiently adapted to target the growing number of human and animal coronavirus strains. Coronavirus infection in populations is characterized by a variety of virologic and serologic states that may not be associated with clinical disease. For example, virus shedding can vary in duration and may be episodic or persistent. Seropositivity does not predict virus shedding and may not persist once virus is cleared, so reinfection is possible. The outcome is that coronaviruses have adapted to continually chum within susceptible populations and under the right conditions can cause epidemic outbreaks.

Because the viral spike protein is responsible for binding the host cell receptor and because neutralizing antibodies against spike have been demonstrated, most coronavirus vaccines have targeted the spike protein as the key immunogen. Unfortunately, spike proteins, including the host receptor binding domain (RBD) also induces antibodies that can enhance infection and accelerate disease. This is termed antibody-dependent enhancement (ADE) and is primarily mediated by IgG binding to Fcγ receptors. To avoid ADE, and among other advantages, the vaccine platform of the present disclosure targets the virus-host cell interaction, which depends on protease cleavage of the viral spike protein at two locations (S 1/S2 and S2′). Preventing cleavage has been shown to reduce virus infectivity by preventing exposure of the RBD and the viral fusion peptide. Additionally, the vaccine platform of the present disclosure can be used to target the viral membrane protein to create additional steric hinderance of virus/host cell binding and potentially induce intracellular virus neutralization. Because coronaviruses are transmitted at mucosal surfaces, immunization via a mucosal route is required to induce a robust mucosal immune response that can provide protection. The humoral and cellular mucosal and systemic immune response in cats infected with feline enteric coronavirus has been characterized. Mucosal IgA was the clearest correlate of protection and included specific responses against V1/V2 and V2′ peptides. Importantly, IgA does not induce ADE because it does not bind the FcγR. Based on this premise, the vaccine platform technology of the present disclosure was used to generate a novel orally-delivered vaccine against SARS-CoV-2. As described further herein, the vaccine platform of the present disclosure exploits aspects of commensal microorganism (e.g., Lactobacillus acidophilus) as an orally-delivered vaccine delivery platform to rapidly induce strong mucosal IgA responses to reduce or prevent viral infection and replication at the respiratory and intestinal mucosa. Induction of systemic IgG and cell-mediated responses will provide coverage for virus that breaches the mucosal barrier. Importantly, a L. acidophilus-based vaccine platform, for example, can be easily adapted to novel emerging coronaviruses and can be rapidly and inexpensively manufactured. It can be easily deployed because it does not require cold-chain and does not require trained medical personnel for administration, which are important advantages of the vaccine platforms of the present disclosure over currently available technology.

To date, coronavirus vaccine strategies have typically employed parental immunization to induce systemic neutralizing antibodies. Nearly all characterized neutralizing antibodies recognize the viral spike protein (S) and because of this, it has been a common approach to include the S protein in vaccine formulations. This approach has typically failed to protect humans or animals and has often resulted in antibody-dependent enhancement of infection (ADE). These results are not surprising because it has been shown that neutralizing epitopes on S are synonymous with enhancing epitopes and the same antibody that might be neutralizing at high concentrations may be enhancing at lower concentrations. The preponderance of data from studies of natural infections demonstrates that it can be counter-productive to include the full-length S in a vaccine strategy.

There are three structural proteins available for antibody recognition on the surface of SARS-CoV-2 particles: S, membrane (M) and envelope (E). E is expressed at low levels and mutations in E of SARS (a closely related human coronavirus) do not affect viral replication. Since immune-escape is possible, E is unlikely to be a useful immunogen. On the other hand, M is highly expressed, is essential for virus assembly and has been shown to suppress the type 1 interferon innate immune response. In support of coronavirus M protein as a vaccine immunogen, previous studies have identified eleven Th1 epitope-containing peptides and two linear antibody binding peptides in the M protein.

As described herein, there are serious concerns with using full-length or large parts (particularly 51) of spike protein for vaccination. However, there are critical protease cleavage sites within S that are conserved across the coronavirus family and that may represent an important target for vaccine development. The conformation of the trimeric S of SARS-CoV-2 that allows efficient recognition of the host cell receptor, ACE2, appears to depend on a furin cleavage site at S1/S2. Interestingly, amino acid insertions and changes in this region distinguishes SARS-CoV-2 from SARS-CoV-1 and enables furin cleavage and may be associated with increased efficiency of transmission. A second cleavage site, S2′, is a serine cleavage site that is common to all coronaviruses. Cleavage at S2′ reveals the fusion peptide that engages the host cell membrane to allow for viral entry.

As described further herein, embodiments of the present disclosure include M as well as S peptides that include each of protease cleavage sites (S1/S2 and S2′) in the vaccine platform strategy. Using the feline coronavirus model, previous studies have demonstrated that cats who recovered from infection have mucosal IgA responses against the S1/S2 and S2′ cleavage sites. This is important because this shows that the uncleaved protein is present in vivo and antibodies against these peptides may potentially block cleavage thereby dramatically reducing virus infectivity.

One of the important advantages of the vaccine platform of the present disclosure includes the use of a recombinant Lactobacillus as an oral vaccine vector. That is, this alternative strategy to parenteral vaccination includes targeting SARS-CoV-2 at the points of transmission and replication: the respiratory and intestinal mucosa. A vaccine strategy that protects the mucosa and the associated initial cellular targets can be critical for protection against SARS-CoV-2 infection and replication. The mucosal immune system is, in many respects, independent of the systemic immune system. For example, 90% of mucosal IgA is produced locally and induction of mucosal immunity is best achieved via mucosal vaccination. While the focus of vaccine testing is often on the induction of neutralizing antibodies, IgA has been shown to protect against viral infections with a broader array of effector functions that include immune exclusion, pathogen aggregation, intracellular neutralization, virus excretion (reverse transcytosis), as well as classical neutralization. Of critical importance, IgA is not known to contribute to ADE because it does not bind the FcγR.

Lactobacilli are an important group of Gram-positive lactic acid bacteria used for food preservation, food bioprocessing, and as probiotics. Lactobacilli are increasingly under investigation as biologic vaccine vectors. Orally delivered lactobacilli survive the hostile environment of the stomach and proximal duodenum due to endogenous acid and bile resistance. Additionally, several cell surface components of the lactobacilli are recognized by immune cells via pattern recognition receptors (PRR). In particular, lipoteichoic acid (LTA), peptidoglycan (PG), and muramyl dipeptide (the subcomponent of PG) are the major immune stimulators recognized by the heterodimeric Toll-like receptor (TLR) 2/6 and nucleotide-binding oligomerization domain 2 (NOD2), respectively. The probiotic strain Lactobacillus acidophilus NCFM is particularly promising as an oral vaccine vector because it is acid and bile tolerant; it expresses mucus-binding proteins and associates with the mucosal epithelium; and it binds to dendritic cells (DCs) through DC-specific intercellular adhesion molecule 3 (ICAM-3)-grabbing nonintegrin (DC-SIGN) and other PRR described above.

Thus, as provided herein, the vaccine platform technology of the present disclosure combines subunit vaccine concepts with probiotic Lactobacillus acidophilus as the vaccine delivery platform. An orally-administered recombinant L. acidophilus vaccine provides many advantages over currently available technology, including, but not limited to the following: a platform that is generally regarded as safe (GRAS) by the FDA; a platform that is relatively inexpensive to manufacture; a platform that is useful to target novel viral antigens to induce both mucosal and systemic immune responses while avoiding ADE; a platform that combines adjuvant strategies shown to enhance mucosal immune responses; and a platform that does not require a cold chain or special training to administer.

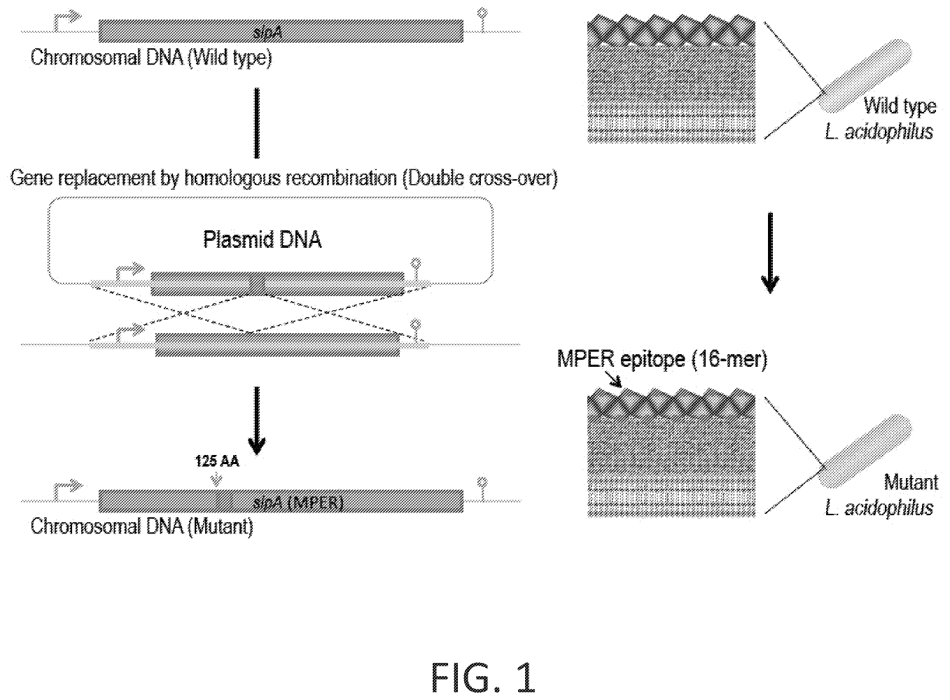

In accordance with these embodiments, and as described further herein, immunogenic or potentially immunogenic polypeptides can be inserted into the surface layer protein (see, e.g., FIG. 1). Full-length membrane protein can be expressed from genomic sequences driven by the L. acidophilus enolase promotor, for example, though other suitable promoters can also be used. All vaccine constructs of the present disclosure were validated by sequencing, western blot, and flow cytometry. Additionally, immunogenicity of the vaccine compositions can be tested in vivo using, for example, C57BL/6J mice immunized with each construct, and immunogenicity can be assessed by ELISA for mucosal IgA and serum IgG. Control groups can be immunized with carrier buffer and/or wild-type LA strain NCK56. In some embodiments, mice can be orally immunized on weeks 0, 4, and 8 with 5×10⁹ rLA in bicarbonate buffer with protease inhibitors as previously described. Post-immunization assessment can be performed as described further herein, except that SARS-CoV-2 peptides and protein will be used for antigen-specific assays. Serum, saliva and fecal antibody can be collected prior to each immunization and 2 weeks post the final immunization. Neutralization assays, virus binding assays, antibody-dependent enhancement assays, protease inhibition assays can be used as a readout for efficacy. At sacrifice, bronchoalveolar lavage, feces, and serum can be collected to assess antigen-specific antibody responses. Pulmonary lymphocytes, Peyer's patches, small intestine mononuclear cells (MNC), mesenteric lymph node and spleen can also be collected for enumeration of SARS-CoV-2-specific antibody-secreting cells (ASC) and antigen-specific T-cell proliferation.

In some embodiments, recombinant SARS-CoV-2 nucleocapsid (recN) protein was produced in E. coli using a 288 residue (aa 133-420) polypeptide with a C-terminal His-tag that contains several B cell epitopes (PMID 27148198). Mice were immunized with recN to generate monoclonal antibodies as well as immunized rabbits for production of polyclonal antibodies. These reagents can be useful for detection of virus in tissues and cell culture experiments described herein. Immune challenges can be performed using human ACE2 (hACE2) transgenic mice (B6.Cg-Tg(K18-ACE2)2Prlmn/J) from the Jackson Laboratory, which were previously shown to develop pulmonary and neurologic disease upon challenge with SARS-CoV.

In some embodiments, dose and pathology of SARS-CoV-2 in hACE2 mice can investigated. An initial challenge experiment can be performed to determine infection kinetics of hACE2 mice that will inform optimal dose for vaccine challenge and permit further characterization of the pathogenesis of the disease. The ID₅₀ and LD₅₀ values of SARS-CoV-2 can be determined by intranasally challenging groups of 5 hACE2 mice with log₁₀ dilutions of virus (e.g., 10² to 10⁶ PFU; 25 inoculated mice plus 5 uninoculated control mice). Mice can be monitored for signs of disease and weighed daily. As mice become moribund, they can be euthanized and necropsied to determine viral loads in organs and tissues (e.g., qPCR and virus titration on Vero E6 cells). Histopathology and viral antigen immunohistochemistry can be performed. Antibody responses can be determined by ELISA and neutralization assays as described further herein.

In accordance with these embodiments, four male and four female mice/group can be immunized as described herein. Negative controls can consist of saline carrier and wild-type L. acidophilus (NCK56). Positive controls can consist of whole, killed SARS-CoV-2 delivered intramuscularly (IM). Because high levels of mucosal IgA have been observed as soon as 2 weeks post the first vaccination with 100% seroconversion after the second immunization using the rLA platform, groups immunized with rLA-SARS-CoV-2 and positive controls can be intranasally-challenged two weeks after being administered 1, 2, or 3 immunizations. This provides an indication of how rapidly protection might be induced during an outbreak situation.

After immunization regimens are completed, the mice can be moved to the ABSL-3 laboratory for infection experiments. Challenge can consist of 50 LD₅₀ of SARS-CoV-2 (isolate 2019-nCoV/USA-WA1-F6/2020) propagated on Vero E6 cells. Mice can be monitored daily for signs of disease and humanely euthanized should they meet institutional euthanasia criteria. On days 2, 4, 7, 10, 14, 21 and 28 post inoculation, nasal, oral and rectal swabs can be collected in surviving mice to quantify viral loads by probe-based real-time PCR assay already in place (WHO test). On days 0, 7, 14, 21 and 28, blood can be collected for serology, including ELISA to viral nucleocapsid (mucosal IgA and systemic IgG) and cytometric imaging for neutralizing antibody. For detection of neutralizing antibodies, serum samples can be diluted 2-fold and incubated with 10³ SARS-CoV-2 in 2% FBS-DMEM at 37° C. for 1 hour. The serum/virus can then be pipetted onto Vero E6 and incubated for 3 days prior to fixation and staining with DAPI and anti-N/AlexaFluor488 to identify infected cells. Plates can be scored for percent infected cells and fluorescent intensity of infected cells (Nexcelom Celigo, requested in budget) to quantify neutralizing antibody response as previously described for MERS-CoV and influenza virus. Enhancing antibody activity can be assessed, for example, as described by Wan et al. using HEK293T cells transfected individually with hACE2 and/or CD32A.

As described further herein, embodiments of the present disclosure provide a vaccine platform technology that can be used to express one or more immunogenic polypeptides (with or without polypeptide adjuvants) to generate a robust immune response against a corresponding pathogenic organism. In addition to demonstrating the use of the vaccine platform of the present disclosure to generate effective vaccine compositions against human and animal coronaviruses, embodiments of the present disclosure also demonstrate use of the vaccine platform to generate effective rotavirus vaccine compositions, which represents a significant obstacle to human health. For example, despite the worldwide licensure and availability of two rotavirus vaccines in 2006, the WHO estimates there continues to be approximately 215,000 rotavirus-related deaths each year. Children under the age of 5 from developing and low-income nations are significantly more likely to be affected by severe rotavirus disease because the currently available vaccines have limited efficacy in these countries compared to developed nations (50-60% versus greater than 85% efficacy). Oral rotavirus vaccine failure in low-income counties has been associated with many factors including malnutrition, environmental enteropathy, and differences in intestinal microbiome composition. Additionally, Rotarix™ and RotaTeq™ are attenuated live virus vaccines that have the risk of excretion of the vaccine strains into the environment, reassortment between the vaccine and wild-type rotavirus strains, and reversion of the vaccine strains to virulence. Both vaccines are contraindicated for use in children with some severe forms of immunodeficiency and have been associated with increased risk of intestinal intussusception, a life-threatening type of intestinal blockage. Rigorous assessment of these disadvantages and decreased efficacy in low-income countries necessitate a new approach for a next generation rotavirus vaccine.

Two proteins, VP7 and VP4, comprise the outer layer of the rotavirus (RV) capsid that interacts with host target cells. Viral infectivity is enhanced by the proteolytic cleavage of VP4 into two proteins designated VP8* and VP5*. To address shortcomings of attenuated live vaccines, subunit vaccines that employ recombinant rotavirus capsid proteins to induce neutralizing antibodies are attracting broad interest. One candidate employing parenteral vaccination with recombinant VP8* protein has reached phase 3 clinical trials. Initial results show mucosal IgA responses have been low most likely due to the parenteral route of immunization and the breadth of neutralizing antibodies has also been limited to homotypic rotavirus strains. This is not surprising given recent data demonstrated that anti-VP8* neutralizing antibodies tend to be homotypic in humans; therefore, it seems clear that multiple capsid proteins will be required for broader (heterotypic) neutralizing antibody responses.

As described herein, embodiments of the present disclosure combine subunit vaccine concepts with probiotic Lactobacillus acidophilus as the vaccine platform for the treatment and/or prevention of rotavirus infections. To address the need for a safe, efficacious, next generation rotavirus vaccine, embodiments of the present disclosure combines the benefits of oral immunization, subunit immunogens, and a probiotic. This concept stands in contrast to the current live attenuated vaccines and the parenteral, single protein subunit vaccine that is in clinical trials. The novel approach to generating a rotavirus vaccine platform includes combining multiple rotavirus capsid peptide and protein immunogens that are rigorously validated based on antibody effector function and protection against challenge. Because of the novelty of the vaccine platform of the present disclosure, a unique approach was used to assess safety with respect to the intestinal microbiome. As described further herein, this is the first evaluation of a recombinant probiotic vaccine platform using the gnotobiotic pig model transplanted with a human infant fecal microbiome. Additionally, constructs can be generated using a novel CRISPR-SpyCas9n system that has been adapted specifically for use in L. acidophilus. This system can be used to assess homotypic and heterotypic neutralizing antibody responses with a newly developed, highly sensitive assay based on HT-29 cells. The mechanism of vaccine-associated shifts in the intestinal microbial community structure can be investigated by determining the magnitude and nature of microbiota-specific mucosal IgA responses.

Rotavirus is a double-stranded non-enveloped RNA virus that is transmitted via the fecal-oral route. Infection generally occurs before the age of 5. The virus infects villous enterocytes in the small intestine causing severe gastroenteritis resulting in decreased intestinal absorption and an isotonic diarrhea leading to dehydration and possibly death. Rotavirus consists of six structural proteins. The capsid is formed by VP4, VP6, and VP7. VP4 is proteolytically cleaved into VP5* and VP8*. VP8* forms the head of the VP4 spike, interacts with host cell receptors and is therefore required for infection. VP4 and VP7 define virus serotype and induce protective neutralizing antibodies whereas VP6 is the most immunodominant of the capsid proteins but has only been shown to induce neutralizing antibodies or protective responses in mice with no protection in other species. Currently licensed rotavirus vaccines (Rotarix™, RotaTeq™), are attenuated live oral vaccines derived from human rotavirus or reassortments of human and bovine rotavirus strains, and thus contain all viral antigens.

Correlates of protection induced by natural infection are incompletely understood. From the perspective of prevention, it has been shown that neutralizing antibodies against the structural capsid proteins prevent infection of enterocytes. Indeed, current attenuated live vaccines provide protection from infection via neutralizing antibodies to VP4 and VP7. Over the past 2 decades, a few highly conserved, specific immunogenic epitopes that can provide protection against rotaviral infection have been identified. These epitopes include linear fragments from VP7 and the trypsin-cleavage fragment VP8*. Use of a subunit vaccine employing well-conserved neutralizing peptides in combination with full-length capsid proteins would address the inherent risks of attenuated live vaccines and focus the immune response on the production of protective neutralizing antibodies.

IgA is essential for preventing rotavirus infection in animal models. In humans, serum and fecal IgA titers have a positive correlation with protection from infection and clinical disease. In a recent study, the human anti-rotavirus IgA was cloned from resident small intestinal B-cells. IgAs that recognized VP4, VP5* and VP7 tended to have heterotypic neutralizing capability whereas fewer IgAs against VP8* were neutralizing and were generally homotypic. In a follow up study, which describes a more sensitive neutralizing assay system, most of the IgAs specific for VP8* were ultimately shown to be neutralizing. These results indicate that protection against human RV is the result of heterotypic and homotypic neutralizing IgA localized to the intestinal mucosa. Due to the essential need for induction of IgA, the method and route of vaccine delivery must be carefully considered, as IgA is best induced locally at mucosal-associated lymphoid tissues (MALT), such as Peyer's patches and mesenteric lymph nodes. The ability to target MALT, stimulate a strong innate immune response, and present immunogenic peptides and proteins makes the oral vaccine compositions of the present disclosure, which are based on use of Lactobacillus acidophilus, highly advantageous as a next generation human rotavirus vaccine strategy.

Embodiments of the present disclosure use a recombinant Lactobacillus as an oral rotavirus vaccine delivery vector. Additionally, as described herein, adjuvants and rotavirus epitopes were constructed and expressed in L. acidophilus. Previous studies have established an upp-based, marker-less, double-crossover homologous recombination-based gene replacement system with counter-selection in L. acidophilus. This method has been used successfully for generating recombinant strains with single or multiple chromosomal deletions or insertions. The system was also employed to construct the recombinant L. acidophilus strain expressing and displaying HIV-1 gp41 membrane proximal external region (MPER) peptides within the L. acidophilus surface layer protein A (SlpA). This system has been used to express a highly conserved neutralizing VP8* epitope along with the adjuvants FimH and FliC.

In accordance with these embodiments, a CRISPR-SpyCas9n based genome editing system was developed for site-directed chromosomal deletion and insertion in L. acidophilus. Despite the lack of an active endogenous CRISPR-Cas system in L. acidophilus, this system exploited the established functionality and wide host-range of the Streptococcus pyogenes Cas9 nickase variant (SpyCas9n, aka SpyCas9^{10DA}) for genome editing in L. acidophilus and other lactobacilli. This is important because the RuvC nickase domain of SpyCas9n is inactivating, leading to nicking of the target sequence rather than genesis of a double-stranded DNA break, which is often lethal in bacteria. This unique CRISPR-SpyCas9n editing system facilitates, expedites and simplifies the procedures for engineering sophisticated recombinant Lactobacillus acidophilus (rLA) for use as an orally-delivered mucosal vaccine platform against human rotavirus.

While it is clear that the microbiome greatly influences the state of mucosal health, how it influences, and is influenced by, the mucosal immune response to vaccines is poorly understood. There is increasing evidence that the immunogenicity and efficacy of the current attenuated live rotavirus vaccines within an individual is related to the intestinal microbiome. Multiple studies have also shown that probiotic administration (e.g., Lactobacillus acidophilus, L. reuteri, L. lactis, L. rhamnosus, Bifidobacterium lactis, B. longum) prior to or at the time of vaccination enhances antibody and B cell responses and provides the mucosa with direct protection from rotavirus infection through interactions with the innate immune system.

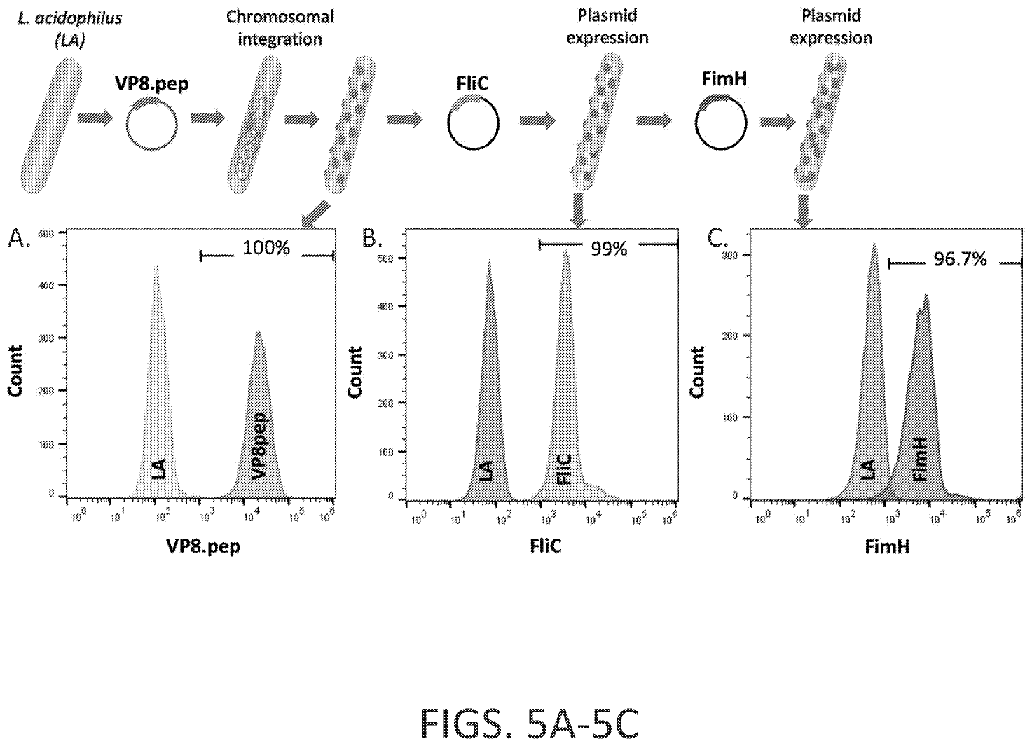

As provided further herein, experiments were conducted to assess immunogenicity and efficacy of mouse VP5*, VP8* and VP7 constructs in the adult mouse/rotavirus model. For this in vivo study, rLA was be engineered to express murine rotavirus strain EC proteins and peptides (FIG. 5). The VP8* and VP7 peptides are highly conserved across rotavirus isolates that infect many species (including human) and the same sequences were used. Peptides were inserted into the surface layer protein (FIG. 5). Full-length proteins were expressed from a plasmid expression system previously used with HIV Gag protein. All constructs were validated by sequencing, western blot and flow cytometry. Eight mice (4 male and 4 female) were immunized with each construct and immunogenicity were assessed by ELISA for mucosal IgA and serum IgG. Control groups were immunized with carrier buffer or wild-type LA strain NCK56. Mice were immunized on weeks 0, 4, and 8 by gavage with 5×10⁹ rLA in bicarbonate buffer with protease inhibitors as previously described. This dose provides the maximum duration of fecal shedding (up to 72 hours) and can be reliably delivered in 100 μl by gavage. Mice were orally challenged with 10⁴ ID₅₀ homotypic mouse rotavirus EDIM (Epizootic Diarrhea of Infant Mice) strain EC two weeks after the third immunization. While adult BALB/c mice do not exhibit clinical disease following rotavirus challenge, it has been established that fecal virus shedding corresponds to infection and provides an excellent model to study anti-rotavirus immunity.

In accordance with the above, experiments can be performed to assess heterotypic and homotypic immunogenicity and antibody efficacy of human VP5*, VP8* and VP7 constructs in neonatal mice. These experiments can determine immunogenicity and efficacy of novel recombinant Lactobacillus acidophilus vaccine constructs expressing human rotavirus capsid proteins VP5*, VP8*, or VP7. These can be evaluated individually so as to rigorously demonstrate the role of each protein in inducing neutralizing antibody. Immunogenicity can be based on rotavirus antigen-specific mucosal (fecal) IgA and serum IgG. rLA constructs can be engineered with human rotavirus antigens along with FliC and FimH to test the breadth of neutralizing antibody. Similarly, groups of 8 mice each (4 male and 4 female) can be immunized with buffer only, wild-type NCK56, rLA-FliC.FimH, rLA-FliC.FimH.VP5, rLA-FliC.FimH.VP8, or rLA-FliC.FimH.VP7. Neonatal mice can be immunized 3 times at 4-week intervals. Neonatal mice can be used to replicate the human vaccination schedule where infants are typically first immunized against rotavirus at 6 weeks of age (then boosted 4 and 8 weeks later depending on the vaccine used), when the intestinal microbiome is not yet developed. Serum and fecal antibody can be collected prior to each immunization and 2 weeks post the final immunization. Neutralization assays can be the readout for efficacy and can be performed to determine titer and breadth of neutralization using MA104 cells and a panel of human rotavirus strains as described by Nair et al., and as well as with HT-29 cells.

Experiments can also be conducted to assess rLA vaccine colonization, shedding and off-target effects on the microbiome. Oral immunization with rLA could influence the intestinal microbiome by direct or indirect mechanisms. L. acidophilus is a lactic acid producing commensal bacterium commonly used as a probiotic. The use of probiotics has been shown to impact the gut microbiome as a result of direct competition for resources with the resident microbiota or indirectly through enhancement of barrier function or immune modulation. Experiments can be conducted to assess if recombinant LA establishes persistent colonization of the gut thereby contributing to a permanent change in the gut microbial community structure.

Experiments can also be conducted to measure the putative differential impact of the immune response on the resident microbiome by monitoring the change of the IgA coated (IgA+) microbiota, including lactobacillus taxa, that are most likely impacted due to their direct interaction with the immune system. Furthermore, experiments can be conducted to test for alterations in the total microbial community structure and the uncoated (IgA-) microbiota. Sequencing of the IgA+ fraction and accurate classification of the results will allow for the identification of those taxa that could be evolutionarily close to L. acidophilus, potentially resulting in their immune targeting. Those taxa that are putatively functionally similar to rLA within the IgA+ microbes can be inferred using PICRUSt. Reduction of IgA+ taxa over time could indicate either an act of the immune response or an act of competition of rLA against its evolutionarily and functionally close relatives. The IgA-microbiome can be used to ascertain whether the relative reduction in binding, if any, is associated with a reduction in the relative presence of the same or similar taxa that are not coated. This will help assess if competition rather than immune response is in action. Reduction of the relative abundance of these taxa overtime may provide more support for competition instead. The total microbiome can be used to further assess the fluctuation of these and other taxa that might not be directly affected by the immune response. This can be addressed using fluorescent activated cell sorting (FACS) to obtain IgA+ and IgA-bacteria, using 16S rRNA sequencing from the total microbiota, IgA-, and IgA+ communities to assess putative changes. Targeted PCR can be used to assess colonization by vaccine constructs. Fecal antibodies against wild-type LA, FliC, and FimH can be measured directly by ELISA as potential correlates to alterations in the intestinal microbial community.

In accordance with the above description and the examples provided below, embodiments of the present disclosure include engineered bacterial cells comprising an exogenous polypeptide(s), and at least one adjuvant polypeptide, which are expressed by the bacterial cell. In accordance with these embodiments, the expression of the exogenous polypeptide(s) and the at least one adjuvant polypeptide can induce an immune response in a host to treat and/or prevent infection from a pathogenic organism.

In some embodiments, the engineered bacterial cell includes at least one of the following genera: Bacillus, Bacteroides, Bifidobacterium, Brevibacteria, Clostridium, Enterococcus, Escherichia coli, Lactobacillus, Lactococcus, Staphylococcus and Streptococcus. In some embodiments, the engineered bacterial cell includes at least one of the following species: Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus crispatus, Lactobacillus gasseri, Lactobacillus johnsonii, Lactobacillus paracasei, Lactobacillus plantarum, Lactobacillus reuteri, Lactobacillus rhamnosus, Lactobacillus salivarius Lactobacillus fermentum, Lactobacillus delbrueckii, and Lactococcus lactis. In some embodiments, the engineered bacterial cell includes at least one of the following strains: L. acidophilus NCFM, L. acidophilus La-14, L. casei Lc11, L. crispatus NCK 1350, L. crispatus NCK 1351, L. crispatus DNH-429, L. gasseri ATCC 33323, L. gasseri NCK 1338, L. gasseri NCK 1340, L. gasseri NCK 1341, L. gasseri NCK 1342, L. gasseri NCK 1343, L. gasseri Lg-36, L. gasseri NCK2140, L. gasseri NCK2141, L.gasseri JV V03, L. plantarum Lp-115, L. johnsonii NCK948, L. johnsonii NCK957, L. johnsonii NCK964, L. johnsonii NCK979, L. johnsonii NCK1370, L. johnsonii NCK2677, L. johnsonii NCC 533 L. plantarum Lpc-37, L. plantarum Lp115, L. rhamnosus HN001, L. rhamnosus GG, L. rhamnosus Lr-32, L. reuteri 1E1, L. salivarius Ls-33, L. salivarius NCK1352, L. salivarius NCK1355, B. lactis BL-04, B. lactis Bb-02, B. lactis B1-04, B. lactis Bi-07, B. breve Bb-03, B. bifidum Bb-06, B. longum B1-05, B. longum sp infantis Bi-26. In some embodiments, these engineered bacterial strains can be formulated as an oral vaccine composition (e.g., as part of a food composition), and administered to a subject in need thereof.

As would be appreciated by one of ordinary skill in the art based on the present disclosure, the vaccine platform technology described herein can be used with any immunogenic or potentially immunogenic polypeptide, such as those from a pathogenic organism (e.g., bacteria, fungi, virus, and the like). In some embodiments, the exogenous polypeptide (e.g., exogenous to the engineered bacterial cell) is expressed on the surface of the bacterial cell, expressed in the cytosol of the bacterial cell, and/or secreted by the bacterial cell. Means for expressing polypeptides encoded by polynucleotides in bacteria are known in the art, and can include the use of promoters and other regulatory elements. In some embodiments, the exogenous polypeptide is a rotavirus antigen. In some embodiments, the rotavirus antigen is selected from the group consisting of VP3, VP4, VP5, VP6, VP7, VP8, and any derivatives or fragments thereof. In some embodiments, the exogenous polypeptide is a coronavirus antigen. In some embodiments, the exogenous polypeptide is a SARS-CoV-2 antigen. In some embodiments, the exogenous polypeptide is a feline enteric coronavirus antigen. In some embodiments, the coronavirus antigen is derived from a nucleocapsid protein (N), a spike protein (S), an envelope protein (E), or a membrane protein (M). In some embodiments, the coronavirus antigen is derived from a non-structural protein, such as one or more of the 16 nonstructural coronavirus proteins (e.g., proteases, RNA-dependent RNA polymerases, a nuclease, helicase, and methyltransferase). Additionally, as would be recognized by one of ordinary skill in the art based on the present disclosure, antigens and epitopes from a coronavirus can be identified based on sequence similarities to epitopes identified in other coronaviruses, and used in conjunction with the various embodiments of the present disclosure (e.g., using Immune Epitope Database and Analysis Resource (IEDB), as described in Grifoni, A. et al., Cell Press (2020): https://marlin-prod.literatumonline.com/pb-assets/journals/research/cell-host-microbe/PDFs/CHOM_2264_S50. pdf).

In some embodiments, the vaccine platform technology of the present disclosure includes co-expressing at least one adjuvant polypeptide with the immunogenic polypeptide, which enhances immunogenicity of the polypeptide. In some embodiments, the at least one adjuvant polypeptide targets the bacterial cell to a host cell, activates a cellular pathway in a host cell, and/or mimics a host cell immune factor. In some embodiments, the at least one adjuvant polypeptide is exogenous to the bacterial cell. In some embodiments, the at least one adjuvant polypeptide is endogenous to the bacterial cell. In some embodiments, the at least one adjuvant polypeptide is expressed on the surface of the bacterial cell, expressed in the cytosol of the bacterial cell, and/or secreted by the bacterial cell. In some embodiments, the at least one adjuvant polypeptide is flagellin (FliC) and/or type 1 fimbrin D-mannose specific adhesin protein (FimH), and any derivatives or fragments thereof.

In some embodiments, the at least one adjuvant polypeptide is a pro-inflammatory cytokine. In some embodiments, the at least one adjuvant polypeptide targets a dendritic cell (DC). As would be recognized by one of ordinary skill in the art based on the present disclosure, other adjuvant polypeptides can be identified as enhancing an immune response in a subject, and these can be incorporated into the vaccine platform of the present disclosure. In some embodiments, the engineered bacterial cell further comprises at least a third adjuvant polypeptide. In some embodiments, the engineered bacterial cell further comprises at least a fourth adjuvant polypeptide. In some embodiments, the engineered bacterial cell further comprises at least a fifth adjuvant polypeptide. In some embodiments, the engineered bacterial cell further comprises at least a sixth adjuvant polypeptide. In some embodiments, the engineered bacterial cell further comprises at least a seventh adjuvant polypeptide. In some embodiments, the engineered bacterial cell further comprises at least an eighth adjuvant polypeptide. In some embodiments, the engineered bacterial cell further comprises at least a ninth adjuvant polypeptide. In some embodiments, the engineered bacterial cell further comprises at least a tenth adjuvant polypeptide.

In some embodiments, the immunogenic polypeptide and the at least one adjuvant polypeptide are co-expressed. In some embodiments, the exogenous polypeptide and/or the at least one adjuvant polypeptide are expressed as fusion polypeptides. In some embodiments, the exogenous polypeptide and/or the at least one adjuvant polypeptide are integrated into an S-layer protein (SLP) or an S-layer associated protein (SLAP). Other integration sites can also be used, including those which facilitate expression of the polypeptides in the cytosol and/or the bacterial surface.

In some embodiments, the cell further comprises at least a second exogenous polypeptide. In some embodiments, the exogenous polypeptide and the at least second exogenous polypeptide are from the same organism. In some embodiments, the exogenous polypeptide and the at least second exogenous polypeptide are from different organisms. In some embodiments, the cell further comprises at least a third exogenous polypeptide. In some embodiments, the cell further comprises at least a fourth exogenous polypeptide. In some embodiments, the cell further comprises at least a fifth exogenous polypeptide. In some embodiments, the cell further comprises at least a sixth exogenous polypeptide. In some embodiments, the cell further comprises at least a seventh exogenous polypeptide. In some embodiments, the cell further comprises at least an eighth exogenous polypeptide. In some embodiments, the cell further comprises at least a ninth exogenous polypeptide. In some embodiments, the cell further comprises at least a tenth exogenous polypeptide.

Embodiments of the present disclosure also include methods of inducing an immune response in a subject by administering a composition comprising any of the engineered bacterial cells described herein. In some embodiments, the composition induces a mucosal and systemic immune response against the exogenous polypeptide. In some embodiments, the composition increases immunoglobulin A (IgA) antibodies specific for the exogenous polypeptide. In some embodiments, the composition increases immunoglobulin G (IgG) antibodies specific for the exogenous polypeptide.

The vaccine compositions and methods of the present disclosure can include preparing a pharmaceutical composition with at least one genetically engineered species, strain, or subtype of bacteria described herein, and administering the pharmaceutical composition to a subject in a therapeutically effective amount. In some embodiments, the method may comprise preparing a pharmaceutical composition with at least one genetically engineered species, strain, or subtype of bacteria described herein, along with a recombinant polynucleotide or polypeptide comprising a sequence encoding an immunogenic polypeptide and/or polypeptide adjuvant, and administering the pharmaceutical composition to a subject in a therapeutically effective amount. In some embodiments, the genetically engineered bacteria and/or recombinant immunogenic polypeptides and/or polypeptide adjuvants disclosed herein are administered orally (e.g., as part of a food composition). In some embodiments, the genetically engineered bacteria and/or recombinant immunogenic polypeptides and/or polypeptide adjuvants can be lyophilized in a gel cap and administered orally. In some embodiments, the genetically engineered bacteria and/or recombinant immunogenic polypeptides and/or polypeptide adjuvants can be administered via a feeding tube or gastric shunt. In some embodiments, the genetically engineered bacteria and/or recombinant immunogenic polypeptides and/or polypeptide adjuvants can be administered as part of a food product. In some embodiments, the food product includes, but is not limited to, yogurt, dairy products, milk-based beverages, fruit beverages, hydration beverages, energy beverages, fruit preparations, meal replacement beverages, and the like.

Pharmaceutical compositions comprising the genetically engineered bacteria and/or recombinant immunogenic polypeptides and/or polypeptide adjuvants of the present disclosure may be used to treat, manage, ameliorate, and/or prevent diseases associated with infections from pathogenic organisms, and/or symptom(s) associated with diseases associated with infections from pathogenic organisms. Pharmaceutical compositions of the also include one or more genetically engineered bacteria, and/or one or more recombinant immunogenic polypeptides and/or polypeptide adjuvants, alone or in combination with prophylactic agents, therapeutic agents, and/or and pharmaceutically acceptable carriers.

In some embodiments, the pharmaceutical composition comprises one species, strain, or subtype of bacteria that are engineered to comprise the genetic modifications described herein (e.g., to express one or more immunogenic polypeptides and a polypeptide adjuvant). In some embodiments, the pharmaceutical composition comprises two or more species, strains, and/or subtypes of bacteria that are each engineered to comprise the genetic modifications described herein.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered bacteria and/or recombinant immunogenic polypeptides and/or polypeptide adjuvants described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, immediate-release, pulsatile-release, delayed-release, or sustained release). Suitable dosage amounts for the genetically engineered bacteria may range from about 10⁵ to 10¹² bacteria, e.g., approximately 10⁵ bacteria, approximately 10⁶ bacteria, approximately 10⁷ bacteria, approximately 10⁸ bacteria, approximately 10⁹ bacteria, approximately 10¹⁰ bacteria, approximately 10¹¹ bacteria, or approximately 10¹² bacteria, or more. The compositions, which may comprise any combinations of the genetically engineered bacteria and/or recombinant enzymes described herein, can be administered once or more daily, weekly, or monthly. These compositions may be administered before, during, or following a meal. In some embodiments, these pharmaceutical compositions can be administered before the subject eats a meal. In some embodiments, these pharmaceutical compositions can be administered currently with a meal. In some embodiments, these pharmaceutical compositions can be administered after the subject eats a meal.

The genetically engineered bacteria and/or recombinant immunogenic polypeptides and/or polypeptide adjuvants may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered bacteria and/or recombinant immunogenic polypeptides and/or polypeptide adjuvants disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

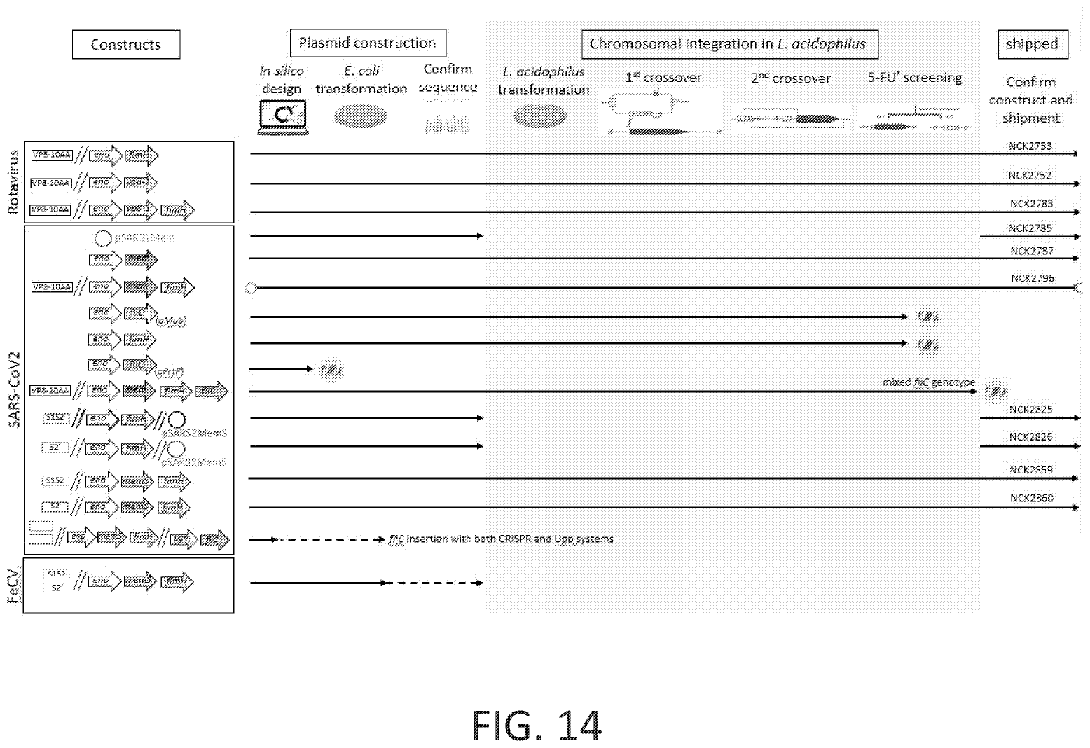

As provided in FIG. 1, various L. acidophilus strains were engineered to express the immunogenic polypeptides and adjuvant polypeptides of the present disclosure. In this exemplary embodiment, polypeptides were expressed within the bacterial surface layer protein after genomic insertion. Viral peptide antigens from HIV-1, human and mouse rotavirus, and human and feline coronaviruses were inserted into the bacterial surface layer protein A (SlpA) using homologous recombination as shown in FIG. 1. The SlpA gene was cloned and the virus peptide was inserted into a region of the sequence known to preserve SlpA structure and provide surface exposure. The plasmid was transfected into L. acidophilus and selected for homologous recombination using nutrient selective pressure. The L. acidophilus bacterial strains containing the polypeptides and adjuvants are provided in Table 1 (below; see also FIG. 14).

TABLE 1
L. acidophilus bacterial strains (MPER: HIV membrane proximal
external region; RV: rotavirus; SARS2: SARS-CoV-2 (COVID-19)).

| Construct nomenclature | Peptide antigen | Polypeptide antigen | Adjuvant |
|---|---|---|---|
| NCK56 (La WT) | None | None | None |
| GAD31 | HIV-1 MPER | None | None |
| GAD10 | HIV-1 MPER | None | FliC |
| GAD40 | HIV-1 MPER | None | FimH |
| rLA.FliC.FimH.S1/S2pep | SARS2 S1/S2 | SARS2 Membrane | FliC and FimH |
| rLA.FliC.FimH.S2′pep | SARS2 S2′ | SARS2 Membrane | FliC and FimH |
| rLA.FliC.FimH.Membrane | | SARS2 Membrane | FliC and FimH |
| rLA.FliC.FimH.VP8pep | RV VP8pep | | FliC and FimH |
| rLA.FliC.FimH.VP8 | | RV VP8 | FliC and FimH |
| rLA.FliC.FimH.VP8.VP8pep | RV VP8pep | RV VP8 | FliC and FimH |
| rLA.FliC.FimH.VP5 | | RV VP5 | FliC and FimH |
| rLA.FliC.FimH.VP7pep | RV VP7pep | | FliC and FimH |
| rLA.FliC.FimH.VP7 | | RV VP7 | FliC and FimH |
| rLA.FliC.FimH.VP7.VP7pep | RV VP7pep | RV VP7 | FliC and FimH |
| rLA.FliC.FimH.FCoV.S1/S2.Mem | FCoV S1/S2 | FCoV Membrane | FliC and FimH |
| rLA.FliC.FimH.FCoV.S2′.Mem | FCoV S2′ | FCoV Membrane | FliC and FimH |
| rLA.FliC.FimH.FCoV.S1/S2.S2 | FCoV S1/S2 | FCoV S2 | FliC and FimH |
| rLA.FliC.FimH.FCoV.S2′.S2 | FCoV S2′ | FCoV S2 | FliC and FimH |

### Example 2

Induction of a robust immune response is key to preventing infection by pathogens, including those that are transmitted at mucosal surfaces (respiratory, gastrointestinal, urogenital). Based on current understanding of the innate immune response, two adjuvant strategies were evaluated as part of the development of a vaccine platform that can be used to generate vaccine compositions expressing any immunogenic polypeptide or potentially immunogenic polypeptide. In this exemplary embodiment, the polypeptide adjuvants tested were FliC and FimH.

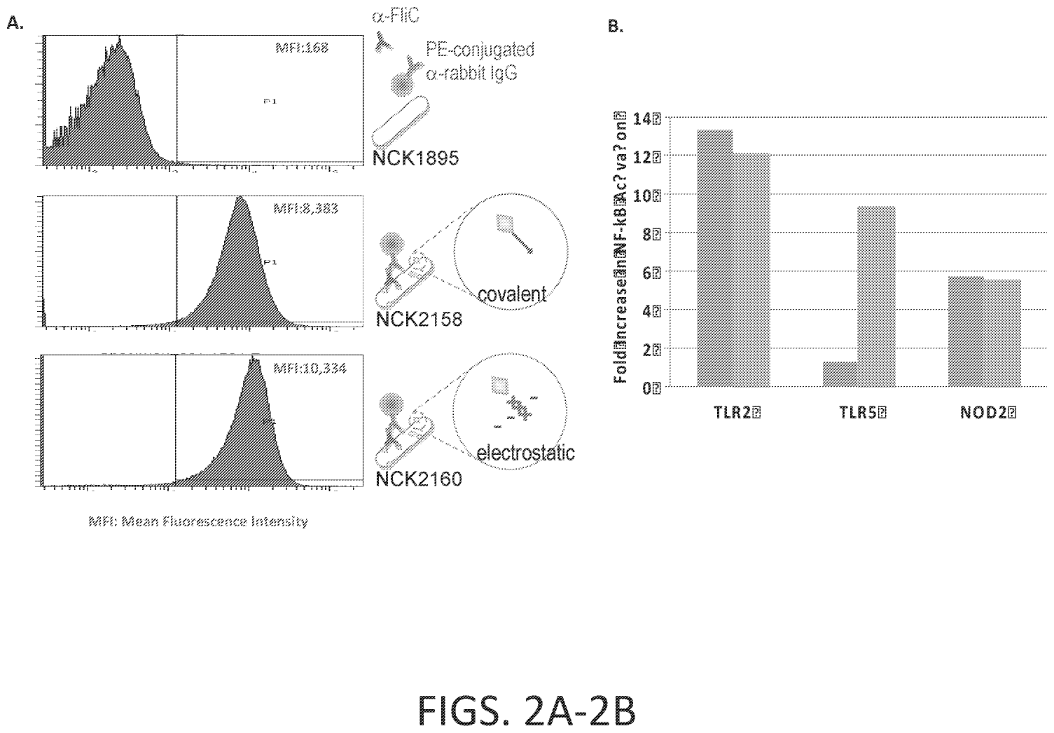

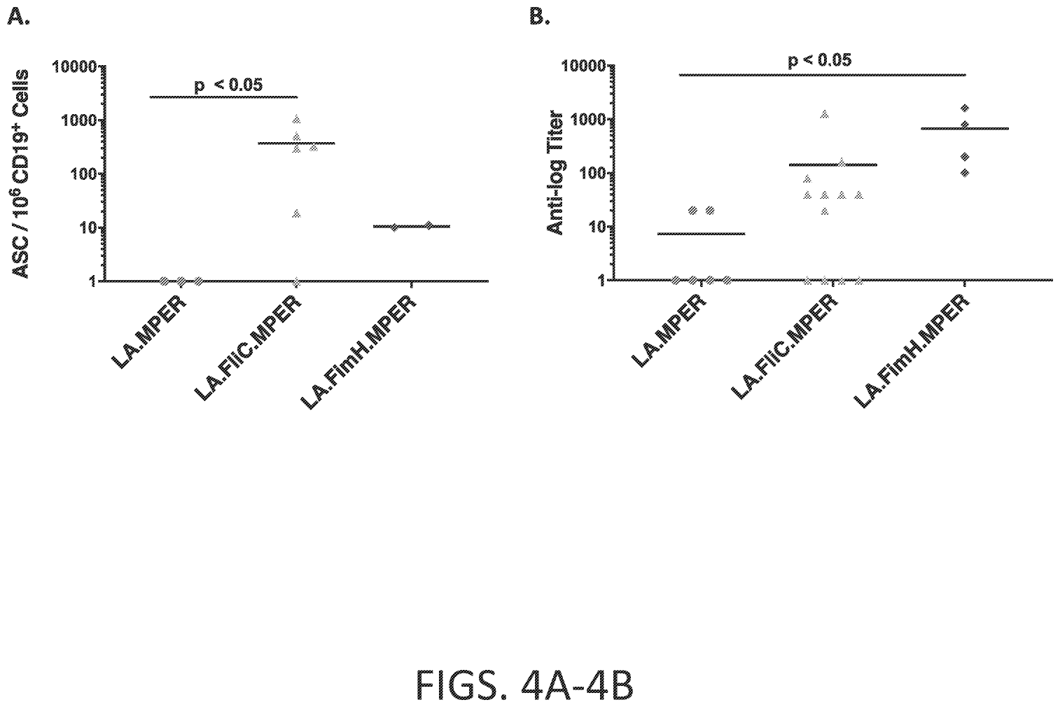

First, experiments were conducted to test the Salmonella typhimurium protein flagellin (FliC), which activates TLR5 and NOD-like receptor C4 (NLRC4). As shown in FIG. 2, surface expression of FliC was verified, along with its biological activity. Additionally, the results in FIG. 2 demonstrated a statistically significant increase in both serum IgG and mucosal IgA against a co-expressed test antigen from Human Immunodeficiency Virus (HIV) termed MPER (FIG. 4).

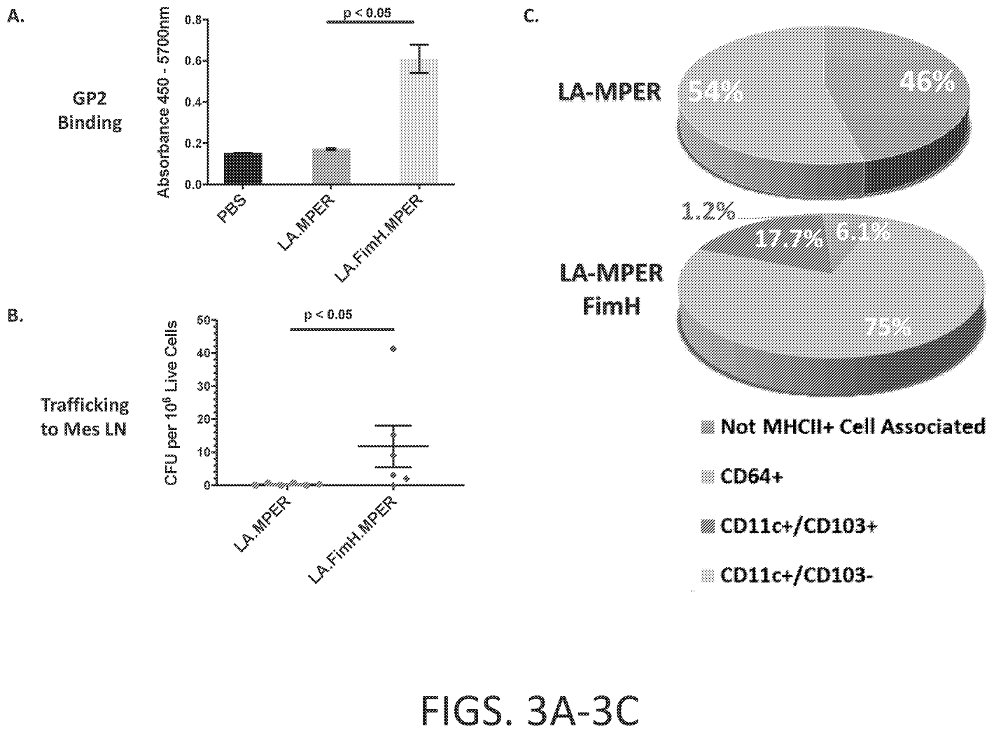

The second adjuvant strategy was designed to increase the uptake of recombinant L. acidophilus (rLA) constructs into immune inductive sites. Antigen uptake in the mucosa occurs via just a few described mechanisms. The major mechanism for sampling particulate antigens is via the follicle associated epithelium known as microfold (M) cells which are associated with Peyer's patches in the oronasal cavity and intestinal tract. M cells are specialized phagocytic cells with blunted villi and an attenuated glycocalyx that allows close interactions of particles with the cell membrane. The type I pilus protein, FimH, is expressed by invasive strains of E. coli and Salmonella spp. and has been shown to bind to glycoprotein 2 (GP2) expressed on M cells. FimH was expressed on the surface of L. acidophilus, and this results in increased translocation from the Peyer's patch to the mesenteric lymph node resulting in increased mucosal IgA producing cells and systemic IgG against co-expressed test antigens (FIGS. 3 and 4).

Taken together, these data demonstrate the feasibility and efficacy of using the L. acidophilus bacterial strains containing the polypeptides and adjuvants of the present disclosure as a vaccine platform technology.

### Example 3

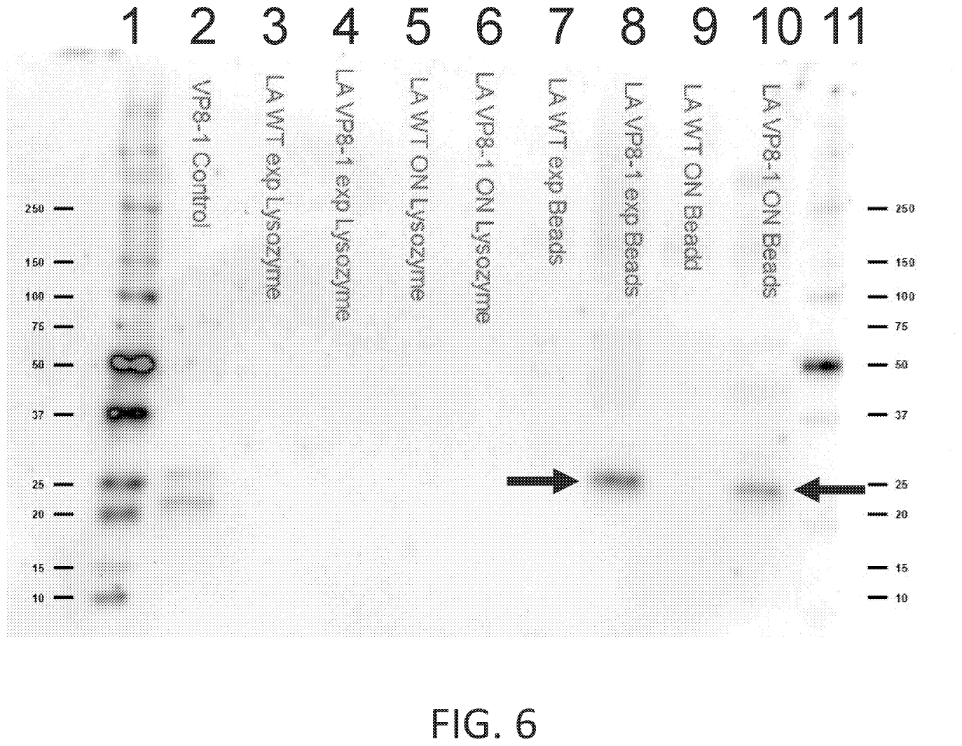

To maximize immunogenic potential, vaccine constructs were developed that express both FliC and FimH, along with immunogenic polypeptides. In this exemplary embodiment, a rotavirus vaccine construct was generated using the vaccine platform of the present disclosure. This construct produced bacterial cells with surface expression of adjuvants (FliC and FimH) and VP8pep (amino acids 1-10), and cytosolic expression of the near full length VP8-1 protein. Surface expression of recombinant proteins was validated by flow cytometry (FIG. 5). Cytosolic expression of VP8-1 (AA 26-231) was validated by western blot (FIG. 6).

### Example 4

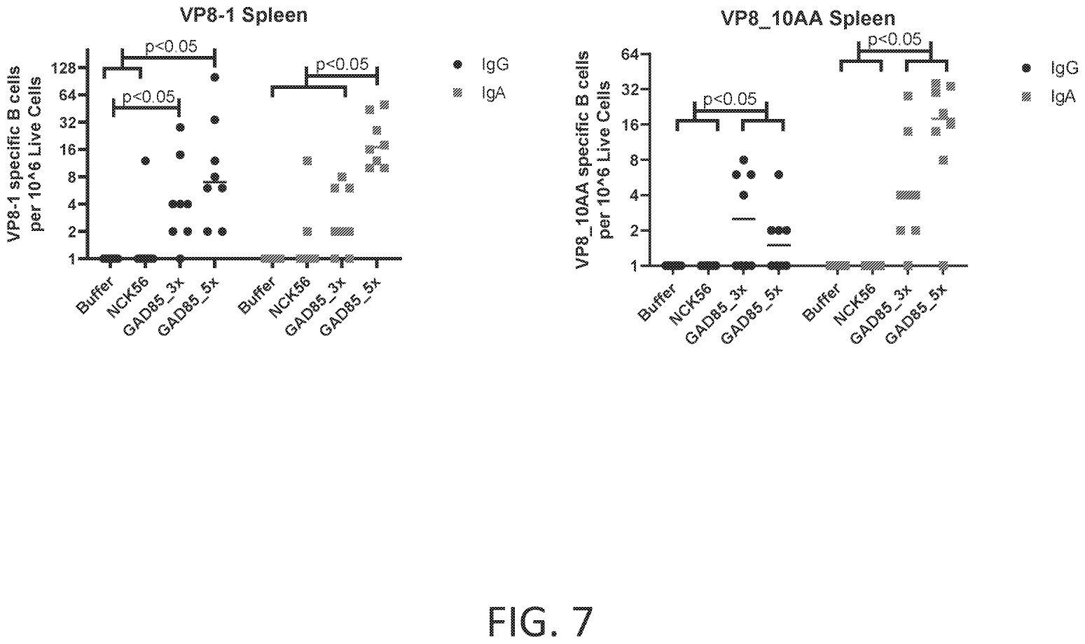

Experiments were then conducted to determine the immunogenicity of the GAD85 construct (Table 1). In this exemplary embodiment, female and male Balb/cJ mice were orally dosed with NCK56 (L. acidophilus without adjuvants and epitopes) or 3 or 5 times with GAD85 (L. acidophilus expressing VP8_10AA in the surface layer protein slpA, VP8-1 in the cytoplasm, and FimH and FliC on the surface). ELISPOT assays revealed a significant increase in splenic VP8-1 and VP8_10AA specific IgA and IgG expressing B cells in mice dosed with GAD85 (FIG. 7). There are significant increases (p<0.05) in VP8_10 AA and VP8-1 specific IgG and IgA B-cells between the mice dosed with GAD85 and the buffer and NCK85 groups.

### Example 5

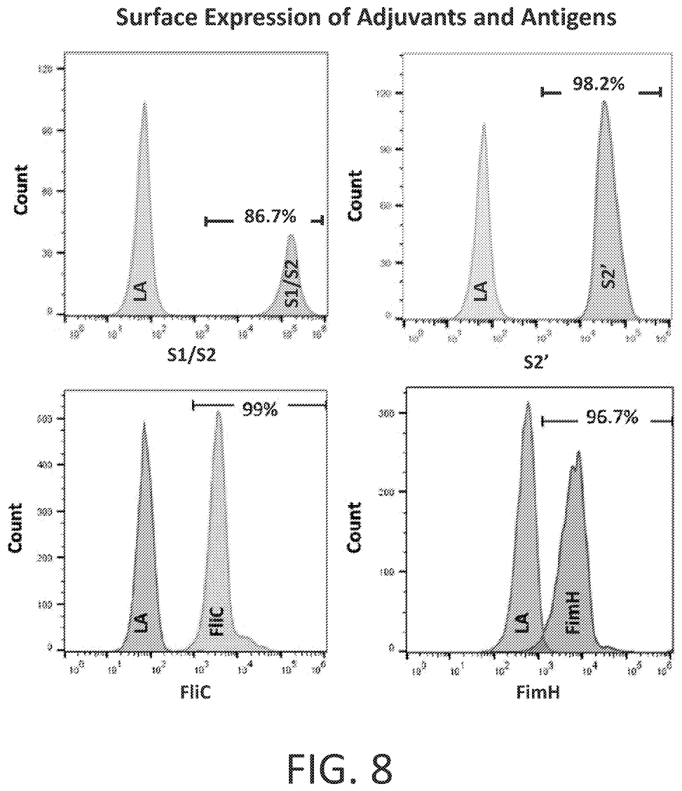

Experiments were also conducted to determine the immunogenicity of constructs containing coronavirus antigens (SARS-CoV-2 antigens). Similar to the rotavirus constructs described above, SARS-CoV-2 peptides, S1/S2 and S2′, were integrated into the genome and their expression was validated on the bacterial surface (FIG. 8). The SARS-CoV-2 membrane (M) protein was also inserted into the bacterial genome and intracellular expression was demonstrated by western blot and mass spec analysis (data not shown). Both FimH and FliC are expressed on the bacterial surface as described above. Mice were immunized as described above and coronavirus antigen-specific fecal IgA and serum IgG was detected in 25-75% of immunized animals depending on the antigen (Table 2).

TABLE 2
Seroconversion of mice immunized
with coronavirus rLA constructs.

| Sample | Percent recognizing S1/S2 | Percent recognizing S2′ |
|---|---|---|
| Fecal IgA | 50% | 50% |
| Serum IgG | 25% | 75% |

Taken together, these data demonstrate the feasibility of using the vaccine platform of the present disclosure to generate a L. acidophilus oral vaccine composition that targets any pathogenic organism. Exemplary pathogenic organisms include, without limitation, the influenza virus, norovirus, non-polio enteric viruses, papillomavirus, respiratory syncytial virus, parainfluenza viruses, rhinovirus, metapneumovirus, and bocaviruses.

### Example 6

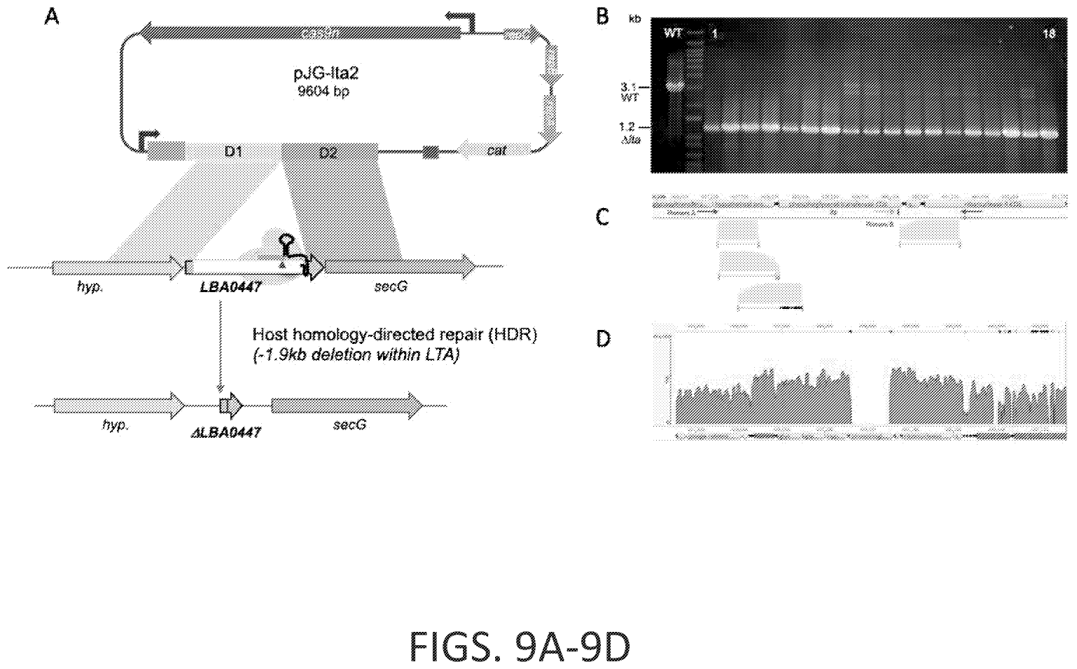

This exemplary embodiment is based on the L. acidophilus-adapted CRISPR-SpyCas9n (SpyCas9^{D10A} RuvC nickase mutant) system on a pORI-derived backbone carrying a chloramphenicol-resistance marker (CAT) for screening, the programmable sgRNA defining the target sequence for editing, and a homologous recombination template designed to engineer the L. acidophilus genome and generate rLA (FIG. 9A). This system enables rapid integration of the desired immunogen or adjuvant sequence into a S-layer protein (SLP) or a S-layer associated protein (SLAP) of interest for expression on the bacterial surface. The sgRNA drives nicking of the target sequence in the WT L. acidophilus strain (NCK56), and the repair template drives homologous recombination of the engineered sequence in frame into the coding sequence of the bacterial protein of interest. PCR-based screening of the chromosomal sequence was then performed to show loss of the WT band and appearance of the edited insert. This CRISPR-SpyCas9n system can be used to alter the lipoteichoic acid (LTA) composition of the NCK56 cell surface with 100% efficiency (FIG. 9B). Specifically, results of experiments have demonstrated that in 100% of the screened colonies, 18/18 screened mutants replaced the 2,061nt LBA0447 gene with a 142nt remnant ORF in which 1,919 coding nucleotides have been excised, corresponding to a 93% deletion of the ORF, with expedited and efficient selection of the mutated genotype over the wild type parent.

Once screening was performed, select colonies were characterized to confirm the mutation genotype, and PCR-amplicons corresponding to the junction fragments were sequenced to confirm the generated construct (FIG. 9C). The genome and transcriptome was the re-sequenced to confirm the engineering outcome (deletion of LTA is shown in FIG. 9C, and the loss of the corresponding transcript is shown in FIG. 9D). Overall, these preliminary results show that the CRISPR-SpyCas9^{D10A} system has been adapted to L. acidophilus and can be efficiently used to generate rLA expressing adjuvants and rotavirus antigens.

### Example 7

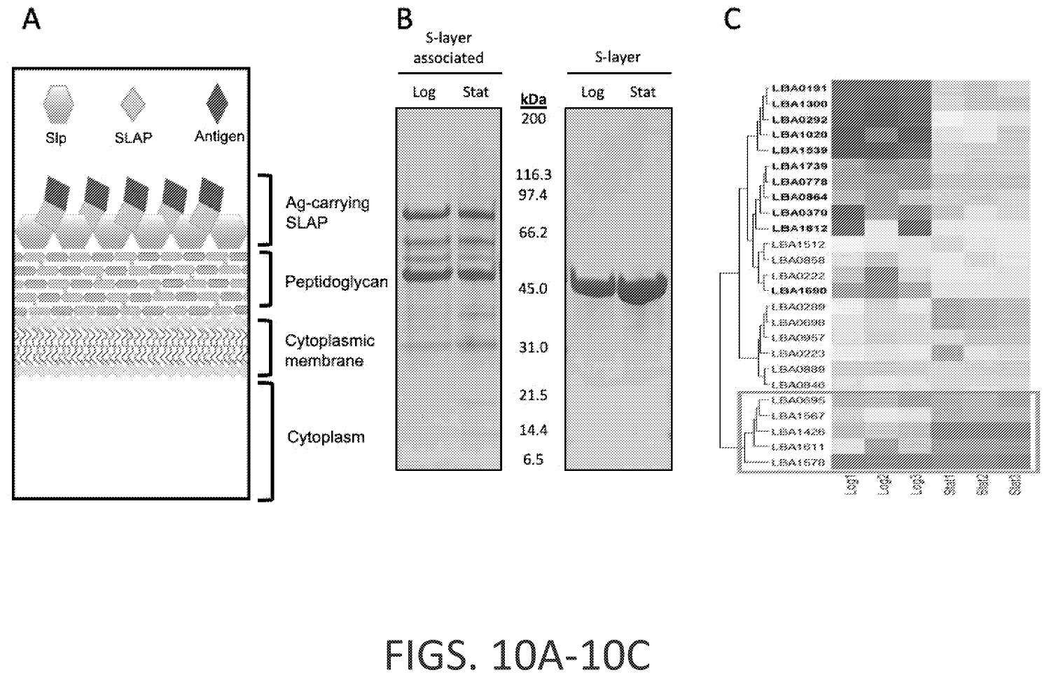

Bacterial cells were engineered to co-express FimH and FliC as separate fusion proteins on the L. acidophilus outer layer with surface-layer associate proteins (SLAPs). These proteins coat the cell surface of the bacilli on top of the peptidoglycan membrane (FIG. 10A), and comprise several individual proteins that associate with the S-layer (FIG. 4B), and vary both in size and relative amounts. Importantly, genomic, proteomic and functional analyses were carried out to determine the composition and relative amounts of the SLAPs expressed by L. acidophilus (FIG. 10C). Comparing and contrasting the SLAPs composition in both log and stationary phases of growth identified five proteins that are present in substantial amounts regardless of the growth phase, including LBA0695, LBA1567, LBA1426, LBA1611, and LBA1578. The location and abundance of these proteins make them ideal candidates for adjuvant and antigen display.

Base constructs were developed that co-express FliC and FimH at levels equal to or greater than that achieved by plasmid expression (FIG. 5). The larger human rotavirus capsid proteins (VP5*, VP7, and VP8*) were expressed, creating 3 individual constructs (rLA.FliC.FimH.VP5, rLA.FliC.FimH.VP8, and rLA.FliC.FimH.VP7) that express these peptides within the surface layer protein as described above. For each adjuvant and/or antigen, insertion fidelity was confirmed as shown in FIG. 10, and surface expression was confirmed by flow cytometry as shown in FIG. 5. Functional activity of FliC and FimH was demonstrated as previously reported for FliC using a TLR5 activation readout (see, e.g., Stoeker, L., et al., Assessment of Lactobacillus gasseri as a candidate oral vaccine vector. Clin Vaccine Immunol, 2011. 18(11): p. 1834-44), and as shown above for FimH (FIG. 4).

### Example 8

In this exemplary embodiment, a 14 amino acid linear epitope sequence from HIV-1 MPER was integrated into the LA genome using upp-based counter-selective gene replacement system (described above) to achieve stable, consistent, and high expression of the epitope of interest within the SlpA (MPER). Sequence integration and antigen expression was confirmed and monitored via PCR, western blot, and flow cytometry. The MPER epitope is a very weak immunogen and there has been limited success in inducing anti-MPER responses, particularly in the mucosa, regardless of the vaccine platform. To overcome this, different adjuvant strategies were used to induce robust mucosal and systemic immune responses against MPER, including plasmid-based expression of secreted mouse IL-1β, and plasmid-based expression of surface-linked Salmonella spp. flagellin (FliC).

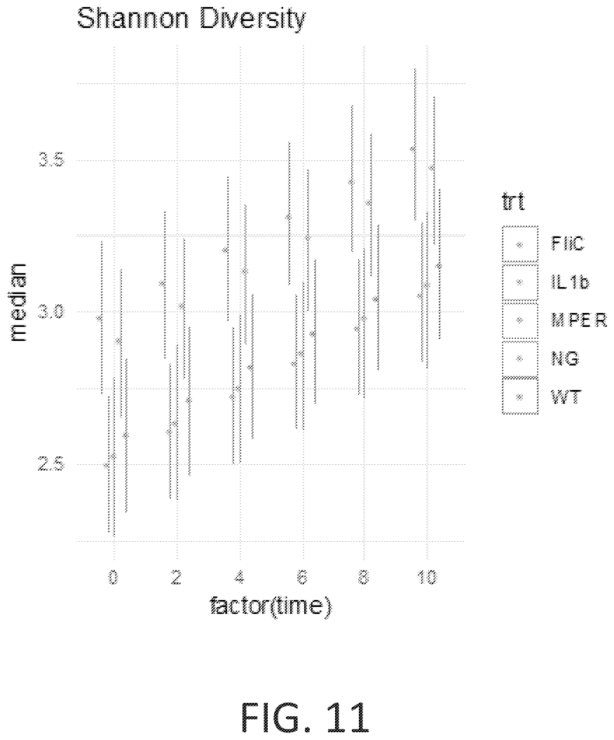

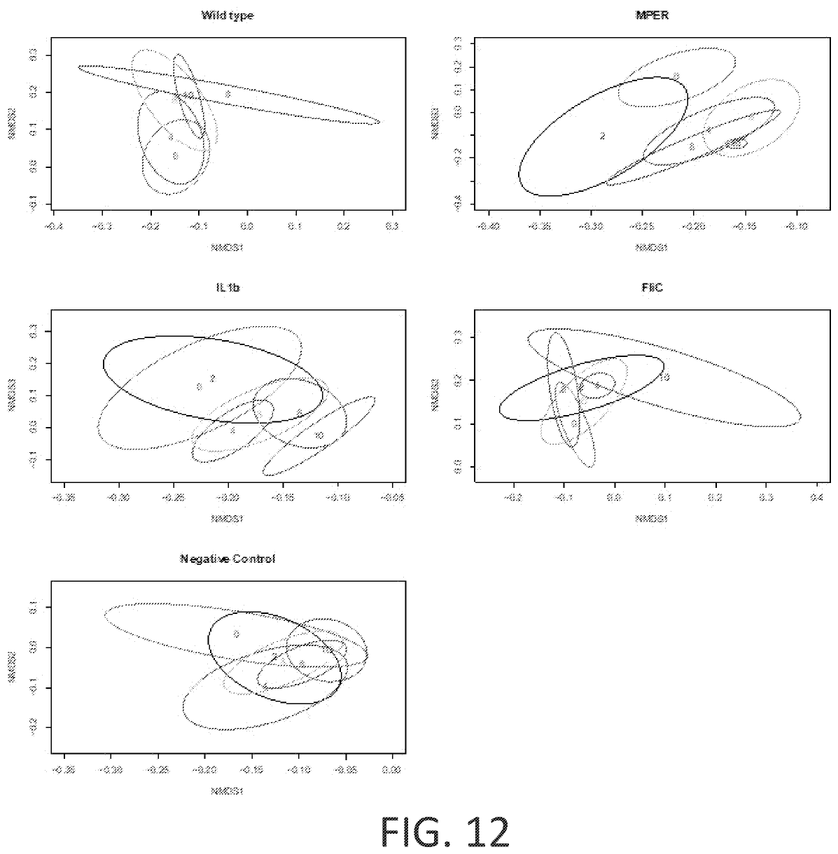

Six animals per group received L. acidophilus wild-type strain (WT), LA-MPER (MPER), LA-MPER secreting IL-1β (IL1b), LA-MPER expressing FliC (FliC), and a negative control group that received only dosing buffer (NG). Results based on Shannon diversity measurements showed that these constructs altered the microbial diversity but not in an identical manner (FIG. 11). While MPER and IL1b constructs have similar effect to the WT positive control, the FliC construct has a similar profile to the negative control. The results also indicate that similar to WT, the MPER and IL1b resulted in a progressive shift in the microbial community structure over time (FIG. 12) that was not observed in association with the negative control (NG) and the FliC construct. These results demonstrated that, depending on the vaccine construct's adjuvant strategy, there can be an impact on the gut microbial community structure. This result suggested the use of FliC as the adjuvant for the proposed studies over IL1β.

### Example 9

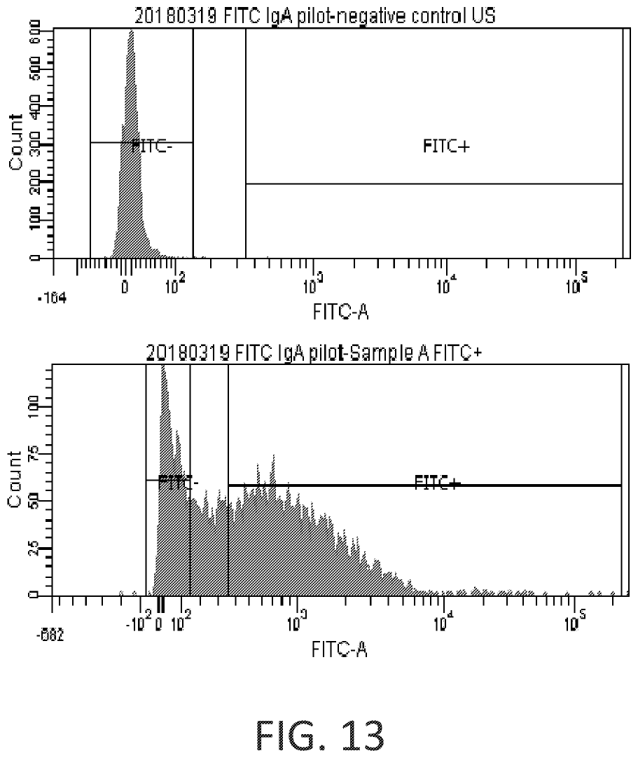

Experiments can then be conducted to screen for IgA coated microbiota. Fecal samples can be homogenized in a saline buffer containing ProteaseArrest to prevent any digestion of antibodies from proteases found in fecal samples. A portion of this suspension can be saved for an unbiased whole-microbiome analysis for each sample. The remaining suspension can be stained with rat anti-species (mouse or pig) IgA with a FITC tag. Samples can be sorted using flow cytometry (e.g., BD FACSAria™ III). The sorted IgA+ and IgA-cells can be sequenced following the same methods described herein. As shown in FIG. 13, results included a negative isotype control and an IgA coated sample.

Sequences. The various embodiments of the present disclosure described herein may include one or more of the sequences referenced below, which can be found in the corresponding sequence listing.

TABLE 3
Peptide/polypeptide sequences

| Peptide/ | |
|---|---|
| Polypeptide | SEQUENCE (SEQ ID NO) |
| HIV-1 MPER | NEQELLELDKWASLWN (SEQ ID NO: 1) |
| SARS2 S1/S2 | KPSKRSFIEDLLFNK (SEQ ID NO: 2) |
| SARS2 S2′ | QTQTNSPRRARSVASQS (SEQ ID NO: 3) |
| SARS2 Membrane | MRLFARTRSMWSFNPETNILLNVPLHGTILTRPLLESELVIGAVILRG |
| | HLRIAGHHLGRCDIKDLPKEITVATSRTLSYYKLGASQRVAGDSGFAA |
| | YSRYRIGNYKLNTDHSSSSDNIALVQLEH (SEQ ID NO: 4) |
| Rotavirus VP8pep | MASLIYRQLL (SEQ ID NO: 5) |
| Rotavirus VP7pep | TEASTQINDG (SEQ ID NO: 6) |
| Rotavirus VP8 | MGAEKTQNVTVNPGPFAQTGYAPANWGPGETNDTTVEPVLDGPYQPIA |
| | FSPPPEYYILLSPTAPGVIAECTNTVNRWIAIIAIEPNVSPTNRTYTL |
| | FGITEQLTVENSSVDKWKFIDFMKTPTTGSYVRYNILLSSTKLCAVAK |
| | HTDNLYSYVGETPTAGQAYYSSFNIFNLTAHCDFYIIPWSQQSLCTQY |
| | VNNGLPPIQNTRNV (SEQ ID NO: 7) |
| Rotavirus VP7 | MYGIEYTTILIFLISIILLNYILKSVTRMMDYIIYRSLLISVALFALT |
| | RAQNYGINLPITGSMDAAYANSTQEGIFLTSTLCLYYPTEASTQINDG |
| | EWKDSLSQMFLIKGWPTGSVYFKEYSSIVDFSVDPQLYCDYNLVLMKY |
| | DQDLELDMSELADLILNEWLCNPMDITLYYYQQSGESNKWISMGSSCT |
| | VKVCPLNTQTLGIGCQTTNVDSFEMVAENEKLAIVDVVDGINHKINLT |
| | TTTCTIRNCKKLGPRENVAVIQVGGSNVLDITADPTTNPQTERMMRVN |
| | WKKWWQVFYTIVDYINQIVQVMSKRSRSLNSAAFYYRV (SEQ ID |
| | NO: 8) |
| Rotavirus VP4 | MASLIYRQLLTNSYSVDLHDEIEQIGSEKTQNVTINPSPFAQTRYAPV |
| | NWGHGEINDSTTVEPILDGPYQPTTFTPPNDYWILINSNTNGVWESTN |
| | NSDFWTAVVAIEPHVNPVDRQYTIFGESKQFNVSNDSNKWKFLEMFRS |
| | SSQNEFYNRRTLTSDTRFVGILKYGGRVWTFHGETPRATTDSSSTANL |
| | NNISITIHSEFYIIPRSQESKCNEYINNGLPPIQNTRNWPLPLSSRSI |
| | QYKRAQVNEDIIVSKTSLWKEMQYNRDIIRFKFGNSIVKMGGLGYKWS |
| | EISYKAANYQYNYLRDGEQVTAHTTCSVNGVNNFSYNGGSLPTDFGIS |
| | RYEVIKENSYVYVDYWDDSKAFRNMVYVRSLAANLNSVKCTGGSYNFS |
| | IPVGAWPVMNGGAVSLHFAGVTLSTQFTDFVSLNSLRFRFSLTVDEPP |
| | FSILRTRTVNLYGLPAANPNNGNEYYEISGRFSLIYLVPTNDDYQTPI |
| | MNSVTVRQDLERQLTDLREEFNSISQEIAMAQLIDLAIIPIDMFSMFS |
| | GIKSTIDLTKSMATSVMKKFRKSKLATSISEMTNSLSDAASSASRNVS |
| | IRSNLSAISNWTNVSNDVSNVTNSLNDISTQTSTISKKFRLKEM1TQT |
| | EGMSFDDISAAVLKTKIDMSTQIGKNTLPDIVTEASEKFIPKRSYRIL |
| | KDDEVMEINTEGKFFAYKINTFDEVPFDVNKFAELVTDSPVISAIIDF |
| | KTLKNLNDNYGITRTEALNLIKSNPNMLRNFINQNNPIIRNRIEQLLL |
| | QCKL (SEQ ID NO: 9) |
| FCoV S1/S2 | KKHQRRSRRSTTTSINTY (SEQ ID NO: 10) |
| FCoV S2′ | PIIGKRSAVEDLLFNKVV (SEQ ID NO: 11) |
| FCoV Membrane | LYRRTKSWWSFNPETNAILCINALGRNYILPLDGTPTGVTLTLLSGNL |
| | YAEGFKMAGGLTIDHLPKYVMIATPSRTIVYTLVGKQLKATTATGWAY |
| | YVKSKAGDYSTEARTDNLSEHEKLLHMV (SEQ ID NO: 12) |
| FCoV S2 | SVASQSIIAYTMSLGAENSVAYSNNSIAIPTNFTISVTTEILPVSMTK |
| | TSVDCTMYICGDSTECSNLLLQYGSFCTQLNRALTGIAVEQDKNTQEV |
| | FAQVKQIYKTPPIKDFGGFNFSQILPDPSKPSKRSFIEDLLFNKVTLA |
| | DAGFIKQYGDCLGDIAARDLICAQKFNGLTVLPPLLTDEMIAQYTSAL |
| | LAGTITSGWTFGAGAALQIPFAMQMAYRFNGIGVTQNVLYENQKLIAN |
| | QFNSAIGKIQDSLSSTASALGKLQDVVNQNAQALNTLVKQLSSNFGAI |
| | SSVLNDILSRLDKVEAEVQIDRLITGRLQSLQTYVTQQLIRAAEIRAS |
| | ANLAATKMSECVLGQSKRVDFCGKGYHLMSFPQSAPHGVVFLHVTYVP |
| | AQEKNFTTAPAICHDGKAHFPREGVFVSNGTHWFVTQRNFYEPQIITT |
| | DNTFVSGNCDVVIGIVNNTVYDPLQPELDSFKEELDKYFKNHTSPDVD |
| | LGDISGINASVVNIQKEIDRLNEVAKNLNESLIDLQELGKYEQYIKWP |
| | W (SEQ ID NO: 13) |
| FliC | MAQVINTNSLSLLTQNNLNKSQSALGTAIERPSSGLRINNAKDDAAGQ |
| | AIANRFTANIKGLTQASRNANDGISIAQTTEGALNEINNNLQRVRELA |
| | VQSANSTNSQSDLDSIQAEITQRLNEIDRVSGQTQFNGVKVLAQDNTL |
| | TIQVGANDGETIDIDLKQINSQTLGLDTLNVQQKYKVSDTAATVTGYA |
| | DTTIALDNSTFKASATGLGGTDQKIDGDLKFDDTTGKYYAKVTVTGGT |
| | GKDGYYEVSVDKTNGEVTLAGGATSPLTGGLPATATEDVKNVQVANAD |
| | LTEAKAALTAAGVTGTASVVKMSYTDNNGKTIDGGLAVKIGDDYYSAT |
| | QNKDGSISINTTKYTADDGTSKTALNKLGGADGKTEVVSIGGKTYAAS |
| | KAEGHNFKAQPDLAEAAATTTENPLQKIDAALAQVDTLRSDLGAVQNR |
| | FNSAITNLGNTVNNLTSARSRIEDSDYATEVSNMSRAQILQQAG |
| | (SEQ ID NO: 14) |
| FimH | FACKTANGTAIPIGGGSANVYVNLAPVVNVGQNLVVDLSTQIFCHNDY |
| | PETIDYVTLQSAYGGVLSNFSGTVKYSGSSYPFPTTSETPRVVYNSRT |
| | DKPWPVALYLTPVSSAGGVAIKAGSLIAVLILRQTNNYNSDDFQFVWN |
| | YIANNDVVVPTGG (SEQ ID NO: 15) |
| FimH.full | FACKTANGTAIPIGGGSANVYVNLAPVVNVGQNLVVDLSTQIFCHNDY |
| | PETITDYVTLQRGSAYGGVLSNFSGTVKYSGSSYPFPTTSETPRVVYN |
| | SRTDKPWPVALYLTPVSSAGVAIKAGSLIAVLILRQTNNYNSDDFQFV |
| | WNIYANNDVVVPTGGCDVSARDVTVTLPDYPGSVPIPLTVYCAKSQNL |
| | GYYLSGTTADAGNSIFTNTASFSPAQGVGVQLTRNGTIIPANNTVSLG |
| | AVGTSAVSLGLTANYARTGGQVTAGNVQSIIGVTFVYQ (SEQ ID |
| | NO: 16) |

What is claimed is:

1. An engineered bacterial cell comprising:
(i) an exogenous polypeptide; and
(ii) at least one adjuvant polypeptide;
wherein the exogenous immunogenic polypeptide and the at least one adjuvant polypeptide are expressed by the bacterial cell.

2. The engineered bacterial cell of claim 1, wherein the cell is selected from at least one of the following genera: Bacillus, Bacteroides, Bifidobacterium, Brevibacteria, Clostridium, Enterococcus, Escherichia coli, Lactobacillus, Lactococcus, Staphylococcus and Streptococcus.

3. The engineered bacterial cell of claim 1 or 2, wherein the cell is selected from at least one of the following species: Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus crispatus, Lactobacillus gasseri, Lactobacillus johnsonii, Lactobacillus paracasei, Lactobacillus plantarum, Lactobacillus reuteri, Lactobacillus rhamnosus, Lactobacillus salivarius Lactobacillus fermentum, Lactobacillus delbrueckii, and Lactococcus lactis.

4. The engineered bacterial cell of any of claims 1 to 3, wherein the cell is selected from at least one of the following strains: L. acidophilus NCFM, L. acidophilus La-14, L. casei Lc11, L. crispatus NCK 1350, L. crispatus NCK 1351, L. crispatus DNH-429, L. gasseri ATCC 33323, L. gasseri NCK 1338, L. gasseri NCK 1340, L. gasseri NCK 1341, L. gasseri NCK 1342, L. gasseri NCK 1343, L. gasseri Lg-36, L. gasseri NCK2140, L. gasseri NCK2141, L.gasseri JV V03, L. plantarum Lp-115, L. johnsonii NCK948, L. johnsonii NCK957, L. johnsonii NCK964, L. johnsonii NCK979, L. johnsonii NCK1370, L. johnsonii NCK2677, L. johnsonii NCC 533 L. plantarum Lpc-37, L. plantarum Lp115, L. rhamnosus HN001, L. rhamnosus GG, L. rhamnosus Lr-32, L. reuteri 1E1, L. salivarius Ls-33, L. salivarius NCK1352, L. salivarius NCK1355, B. lactis BL-04, B. lactis Bb-02, B. lactis Bl-04, B. lactis Bi-07, B. breve Bb-03, B. bifidum Bb-06, B. longum B1-05, B. longum sp infantis Bi-26.

5. The engineered bacterial cell of any of claims 1 to 4, wherein the exogenous polypeptide is immunogenic.

6. The engineered bacterial cell of any of claims 1 to 5, wherein the exogenous polypeptide is expressed on the surface of the bacterial cell, expressed in the cytosol of the bacterial cell, and/or secreted by the bacterial cell.

7. The engineered bacterial cell of any of claims 1 to 6, wherein the exogenous polypeptide is an antigen from a pathogenic organism.

8. The engineered bacterial cell of claim 7, wherein the pathogenic organism is a bacteria or a virus.

9. The engineered bacterial cell of any of claims 1 to 8, wherein the exogenous polypeptide is a rotavirus antigen.

10. The engineered bacterial cell of claim 8, wherein the rotavirus antigen is selected from the group consisting of VP3, VP4, VP5, VP6, VP7, VP8, and any derivatives or fragments thereof.

11. The engineered bacterial cell of any of claims 1 to 8, wherein the exogenous polypeptide is a coronavirus antigen.

12. The engineered bacterial cell of claim 11, wherein the exogenous polypeptide is a SARS-CoV-2 antigen.

13. The engineered bacterial cell of claim 11, wherein the exogenous polypeptide is a feline enteric coronavirus antigen.

14. The engineered bacterial cell of claim 12 or claim 13, wherein the coronavirus antigen is derived from a nucleocapsid protein (N), a spike protein (S), an envelope protein (E), or a membrane protein (M).

15. The engineered bacterial cell of any of claims 1 to 14, wherein the at least one adjuvant polypeptide enhances immunogenicity of the exogenous polypeptide.

16. The engineered bacterial cell of any of claims 1 to 15, wherein the at least one adjuvant polypeptide targets the bacterial cell to a host cell, activates a cellular pathway in a host cell, and/or mimics a host cell immune factor.

17. The engineered bacterial cell of any of claims 1 to 16, wherein the at least one adjuvant polypeptide is exogenous to the bacterial cell.

18. The engineered bacterial cell of any of claims 1 to 16, wherein the at least one adjuvant polypeptide is endogenous to the bacterial cell.

19. The engineered bacterial cell of any of claims 1 to 18, wherein the at least one adjuvant polypeptide is expressed on the surface of the bacterial cell, expressed in the cytosol of the bacterial cell, and/or secreted by the bacterial cell.

20. The engineered bacterial cell of any of claims 1 to 19, wherein the at least one adjuvant polypeptide is flagellin (FliC) and/or type 1 fimbrin D-mannose specific adhesin protein (FimH), and any derivatives or fragments thereof.

21. The engineered bacterial cell of any of claims 1 to 19, wherein the at least one adjuvant polypeptide is a pro-inflammatory cytokine.

22. The engineered bacterial cell of any of claims 1 to 19, wherein the at least one adjuvant polypeptide targets a dendritic cell (DC).

23. The engineered bacterial cell of any of claims 1 to 22, wherein the exogenous polypeptide and the at least one adjuvant polypeptide are co-expressed.

24. The engineered bacterial cell of any of claims 1 to 22, wherein the exogenous polypeptide and/or the at least one adjuvant polypeptide are expressed as fusion polypeptides.

25. The engineered bacterial cell of any of claims 1 to 22, wherein the exogenous polypeptide and/or the at least one adjuvant polypeptide are integrated into an S-layer protein (SLP) or an S-layer associated protein (SLAP).

26. The engineered bacterial cell of any of claims 1 to 25, wherein the cell further comprises at least a second exogenous polypeptide.

27. The engineered bacterial cell of claim 26, wherein the exogenous polypeptide and the at least second exogenous polypeptide are from the same organism.

28. The engineered bacterial cell of claim 27, wherein the exogenous polypeptide and the at least second exogenous polypeptide are from different organisms.

29. A vaccine composition comprising the engineered bacterial cell of any of claims 1 to 28.

30. A food product comprising the engineered bacterial cell of any of claims 1 to 28.

31. A method of inducing an immune response in a subject comprising administering a composition comprising the engineered bacterial cell of any of claims 1 to 28 to the subject.

32. The method of claim 31, wherein the composition induces a mucosal and systemic immune response against the exogenous polypeptide.

33. The method of claim 31 or claim 32, wherein the composition increases immunoglobulin A (IgA) antibodies specific for the exogenous polypeptide.

34. The method of any of claims 31 to 33, wherein the composition increases immunoglobulin G (IgG) antibodies specific for the exogenous polypeptide.


SEQUENCE LISTING (16 sequences)

SEQ ID NO: 1 - protein, 16 aa, Artificial Sequence
  Synthetic
NEQELLELDKWASLWN

SEQ ID NO: 2 - protein, 15 aa, Artificial Sequence
  Synthetic
KPSKRSFIEDLLFNK

SEQ ID NO: 3 - protein, 17 aa, Artificial Sequence
  Synthetic
QTQTNSPRRARSVASQS

SEQ ID NO: 4 - protein, 125 aa, Artificial Sequence
  Synthetic
MRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALVQLEH

SEQ ID NO: 5 - protein, 10 aa, Artificial Sequence
  Synthetic
MASLIYRQLL

SEQ ID NO: 6 - protein, 10 aa, Artificial Sequence
  Synthetic
TEASTQINDG

SEQ ID NO: 7 - protein, 206 aa, Artificial Sequence
  Synthetic
MGAEKTQNVTVNPGPFAQTGYAPANWGPGETNDTTVEPVLDGPYQPIAFSPPPEYYILLSPTAPGVIAECTNTVNRWIAIIAIEPNVSPTNRTYTLFGITEQLTVENSSVDKWKFIDFMKTPTTGSYVRYNILLSSTKLCAVAKHTDNLYSYVGETPTAGQAYYSSFNIFNLTAHCDFYIIPWSQQSLCTQYVNNGLPPIQNTRNV

SEQ ID NO: 8 - protein, 326 aa, Artificial Sequence
  Synthetic
MYGIEYTTILIFLISIILLNYILKSVTRMMDYIIYRSLLISVALFALTRAQNYGINLPITGSMDAAYANSTQEGIFLTSTLCLYYPTEASTQINDGEWKDSLSQMFLIKGWPTGSVYFKEYSSIVDFSVDPQLYCDYNLVLMKYDQDLELDMSELADLILNEWLCNPMDITLYYYQQSGESNKWISMGSSCTVKVCPLNTQTLGIGCQTTNVDSFEMVAENEKLAIVDVVDGINHKINLTTTTCTIRNCKKLGPRENVAVIQVGGSNVLDITADPTTNPQTERMMRVNWKKWWQVFYTIVDYINQIVQVMSKRSRSLNSAAFYYRV

SEQ ID NO: 9 - protein, 775 aa, Artificial Sequence
  Synthetic
MASLIYRQLLTNSYSVDLHDEIEQIGSEKTQNVTINPSPFAQTRYAPVNWGHGEINDSTTVEPILDGPYQPTTFTPPNDYWILINSNTNGVVYESTNNSDFWTAVVAIEPHVNPVDRQYTIFGESKQFNVSNDSNKWKFLEMFRSSSQNEFYNRRTLTSDTRFVGILKYGGRVWTFHGETPRATTDSSSTANLNNISITIHSEFYIIPRSQESKCNEYINNGLPPIQNTRNVVPLPLSSRSIQYKRAQVNEDIIVSKTSLWKEMQYNRDIIIRFKFGNSIVKMGGLGYKWSEISYKAANYQYNYLRDGEQVTAHTTCSVNGVNNFSYNGGSLPTDFGISRYEVIKENSYVYVDYWDDSKAFRNMVYVRSLAANLNSVKCTGGSYNFSIPVGAWPVMNGGAVSLHFAGVTLSTQFTDFVSLNSLRFRFSLTVDEPPFSILRTRTVNLYGLPAANPNNGNEYYEISGRFSLIYLVPTNDDYQTPIMNSVTVRQDLERQLTDLREEFNSLSQEIAMAQLIDLALLPLDMFSMFSGIKSTIDLTKSMATSVMKKFRKSKLATSISEMTNSLSDAASSASRNVSIRSNLSAISNWTNVSNDVSNVTNSLNDISTQTSTISKKFRLKEMITQTEGMSFDDISAAVLKTKIDMSTQIGKNTLPDIVTEASEKFIPKRSYRILKDDEVMEINTEGKFFAYKINTFDEVPFDVNKFAELVTDSPVISAIIDFKTLKNLNDNYGITRTEALNLIKSNPNMLRNFINQNNPIIRNRIEQLILQCKL

SEQ ID NO: 10 - protein, 18 aa, Artificial Sequence
  Synthetic
KKHQRRSRRSTTTSINTY

SEQ ID NO: 11 - protein, 18 aa, Artificial Sequence
  Synthetic
PIIGKRSAVEDLLFNKVV

SEQ ID NO: 12 - protein, 124 aa, Artificial Sequence
  Synthetic
LYRRTKSWWSFNPETNAILCINALGRNYILPLDGTPTGVTLTLLSGNLYAEGFKMAGGLTIDHLPKYVMIATPSRTIVYTLVGKQLKATTATGWAYYVKSKAGDYSTEARTDNLSEHEKLLHMV

SEQ ID NO: 13 - protein, 529 aa, Artificial Sequence
  Synthetic
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPW

SEQ ID NO: 14 - protein, 476 aa, Artificial Sequence
  Synthetic
MAQVINTNSLSLLTQNNLNKSQSALGTAIERPSSGLRINNAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKIGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAG

SEQ ID NO: 15 - protein, 157 aa, Artificial Sequence
  Synthetic
FACKTANGTAIPIGGGSANVYVNLAPVVNVGQNLVVDLSTQIFCHNDYPETIDYVTLQSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGG

SEQ ID NO: 16 - protein, 278 aa, Artificial Sequence
  Synthetic
FACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ